(19) United States
(12) Patent Application Publication
(10) Pub. No.: US 2026/0152565 A1
(43) Pub. Date: Jun. 4, 2026

(54) BCMA VH-ONLY CARS

(71) Applicants: DANA-FARBER CANCER INSTITUTE, INC., Boston, MA (US); HARBOUR BIOMED (SHANGHAI) CO., LTD, Shanghai (CN)

(72) Inventors: Eric Lawrence SMITH, Boston, MA (US); Kartika VENUGOPAL, Boston, MA (US); Yun HE, Boston, MA (US); Lei SHI, Boston, MA (US)

(73) Assignees: DANA-FARBER CANCER INSTITUTE, INC., Boston, MA (US); HARBOUR BIOMED (SHANGHAI) CO., LTD, Shanghai (CN)

(21) Appl. No.: 19/126,353

(22) PCT Filed: Nov. 3, 2023
(86) PCT No.: PCT/US2023/078664

§ 371 Date: May 1, 2025

Related U.S. Application Data
(60) Provisional application No. 63/422,711, filed on Nov. 4, 2022.

(51) Int. Cl. C07K 16/28; A61K 31/192; A61K 31/27; A61K 31/381; A61K 31/4174; A61K 31/4245; A61K 31/454; A61K 31/4965; A61K 31/497; A61K 31/5377; A61K 31/55; A61K 31/573; A61K 31/69; A61K 39/395; A61K 40/11; A61K 40/31; A61K 40/42; A61P 35/00; C12N 5/0783

(52) U.S. Cl. CPC C07K 16/2878; A61K 31/192; A61K 31/27; A61K 31/381; A61K 31/4174; A61K 31/4245; A61K 31/454; A61K 31/4965; A61K 31/497; A61K 31/5377; A61K 31/55; A61K 31/573; A61K 31/69; A61K 39/3955; A61K 40/11; A61K 40/31; A61K 40/4202; A61P 35/00; C12N 5/0636; A61K 2239/48; C07K 2317/24; C07K 2317/524; C07K 2317/526; C07K 2317/73; C07K 2317/92; C07K 2319/02; C12N 2510/00

(57) ABSTRACT

Disclosed are nucleic acids encoding a chimeric antigen receptor comprising an extracellular domain comprising an antigen recognition domain consisting of a fully human single variable heavy (VH) domain that binds a first epitope on B-cell maturation antigen (BCMA), a transmembrane domain, and an intracellular domain comprising a signaling domain.

### RELATED APPLICATIONS

This application is a national stage application, filed under 35 U.S.C. § 371, of International Application No. PCT/US2023/078664, filed Nov. 3, 2023, which claims the benefit of priority under 35 U.S.C. § 119(e) to U.S. Provisional Application No. 63/422,711, filed Nov. 4, 2022, each of which is incorporated herein by reference in its entirety.

### GOVERNMENT SUPPORT CLAUSE

This invention was made with government support under P50 CA100707 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Oct. 23, 2023, is named 52095_766001WO_ST.xml and is 83 KB bytes in size.

### BACKGROUND OF THE DISCLOSURE

Chimeric antigen receptor (CAR) T-cell therapy has been a highly effective cancer therapy, especially for treating B-cell acute lymphoblastic leukemia (ALL) and B-cell lymphomas. Despite advances in cancer treatment with CAR T-cell therapies, patients with hematologic malignancies such as multiple myeloma (MM) and lymphoma still frequently experience relapse.

One factor in relapse after CAR T-cell therapy is anti-CAR immunity. The patient's immune system may recognize the CAR as a foreign antigen, which, for most CAR T-cell therapies, is a murine-derived single-chain variable antibody fragment (scFv). Human anti-mouse antibodies target the murine idiotopes used in CAR T-cell therapies, which leads to their neutralization or clearance of the administered CAR T-cells. Murine scFv can also bind to the CD19 epitope and trigger human leukocyte antigen (HLA)-restricted T-cell-mediated immunoregulatory responses, leading to the diminished persistence of CAR T-cells in vivo and cancer relapse (Wagner et al., Nat. Rev. Clin. Oncol. 18:379-393 (2021)).

Nanobodies, also called VH antibodies, are derived from the variable domain of heavy chain-only antibodies (HCAb), which lack light chains and the first constant CH1 domain within the heavy chain. Camelids (e.g., camels) naturally produce HCAb antibodies. However, camelid-based nanobodies are also susceptible to human immune responses when used in CAR T-cell therapy (Rossotti et al., FEBS J. 289:4304-4327 (2022)).

Therefore, there is a need to deliver effective CAR T-cell therapies with less anti-CAR immune response. Such a system and method of use thereof would reduce CAR T-cell therapy relapse and improve patient outcomes.

### SUMMARY OF THE DISCLOSURE

A first aspect of the present disclosure is directed to a nucleic acid that contains a sequence that encodes a chimeric antigen receptor (CAR), wherein the CAR contains an extracellular domain containing a first antigen recognition domain made up solely (consisting) of a fully human single variable heavy (VH) domain (i.e., a fully human VH-only antigen recognition domain) that binds an epitope on B-cell maturation antigen (BCMA), a transmembrane domain, and an intracellular domain that contains a signaling domain. To date, no evidence exists that fully human, VH-only-based binding proteins could be produced and incorporated into a CAR construct for the treatment of cancer or autoimmune disease.

Another aspect of the present disclosure is directed to an expression vector that contains (e.g., having integrated or cloned therein) the nucleic acid sequence encoding the CAR.

Another aspect of the present disclosure is directed to a genetically modified immune cell that contains the nucleic acid that contains a nucleic acid encoding the CAR.

Another aspect of the present disclosure is directed to a method of producing the genetically modified immune cell or a hematopoietic cell. The method entails introducing the expression vector that contains a nucleic acid encoding the CAR into the cell (i.e., the immune cell or the hematopoietic cell).

Another aspect of the present disclosure is directed to a pharmaceutical composition that contains a therapeutically effective number of the cell expressing the CAR.

Another aspect of the present disclosure is directed to a method of treating cancer or autoimmune disease that involves aberrant BCMA activity. The method entails administering to a subject in need thereof a therapeutically effective number of the genetically modified cells (i.e., the immune cells or the hematopoietic cells) expressing the CAR.

As shown in the working examples herein, the present inventors have found that incorporation of a fully human VH-only antigen recognition domain into a CAR construct allows for efficient transduction into the cells, binding to BCMA, and CAR-specific killing of BCMA-expressing target cells.

### BRIEF DESCRIPTION OF THE DRAWINGS

FIGS. 1A-1D are a set of schematics showing the antibody domain and regions of different antibody types. FIG. 1A is a schematic of a conventual mammalian (e.g., human) antibody with two heavy chains, and two light chains for a total molecular weight of about 150 kDa. FIG. 1B is a schematic of a Camelid HCAbs with two heavy chains for a total molecular weight of about 95 kDa. FIG. 1C is a schematic of the human IgH locus showing recombination and translation to produce heavy chain only antibodies. FIG. 1D is a schematic of a transgene containing both human and mouse sequences in a mouse background which has mouse heavy chain (HC) and x light chain knock outs.

FIGS. 2A-2D are a series of line plots showing that humanized HCAb have good binding activity to human and cynomolgus BCMA cell lines. FIG. 2A is a line plot showing four antibodies binding to HEK 293T-hu BCMA cells. FIG. 2B is a line plot showing two antibodies binding to HEK 293T-hu BCMA cells. FIG. 2C is a line plot showing four antibodies binding to HEK 293T-cyno BCMA cells. FIG. 2D is a line plot showing two antibodies binding to HEK 293T-cyno BCMA cells.

FIG. 3 is a line plot showing humanized HCAb and isotype control (iso) binding to NCI-H9292 cells.

FIG. 4 is a line plot showing that humanized HCAb blocked BAFF binding to BCMA cells.

FIGS. 5A-5C are a series of line plots showing that HCAbs have strong binding affinity. FIG. 5A is a line plot that shows binding of PR000943 to hBCMA-his-biotin (Acro, BCA-H82E4). FIG. 5B is a line plot that shows binding of PR001046 to hBCMA-his-biotin.

FIG. 5C is a line plot that shows binding of PR000274 to hBCMA-his-biotin.

FIGS. 6A-6C is a series of line plots showing that HCAbs have strong binding affinity. FIG. 6A is a line plot that shows binding of PR001035 to hBCMA-his-biotin. FIG. 6B is a line plot that shows binding of PR000940 to hBCMA-his-biotin. FIG. 6C is a line plot that shows binding of PR0000274 to hBCMA-his-biotin.

FIGS. 7A-7J are a set of schematic, photographs, and line plots that show the cytotoxicity properties of VH-only anti-BCMA CAR cells. FIG. 7A is a schematic of the CAR transgene (i.e., nucleic acid), and the isolation, transduction, and killing assay procedure. FIG. 7B is a set of photographs that shows VH-only anti-BCMA CAR cells expanding and killing effector cells. FIGS. 7C-7E are a set of bar graphs of the VH-only anti-BCMA CAR T cell clones quantifying target cell killing. FIGS. 7F-7I are a set of bar graphs showing cytokine profiles of four VH-only anti-BCMA CAR T cell clones after co-culture with BCMA-expressing target cells. FIG. 7J is a bar graph of comparative cytokine secretion of the VH-only anti-BCMA CAR T cell clones.

FIGS. 8A-8E are a set of schematics, flow cytometry, and bar plots that show low levels of tonic signaling in VH-only anti-BCMA CAR cells. FIG. 8A is a schematic illustrating the detection of tonic signaling. FIG. 8B is a set of flow cytometry plots showing low levels of tonic signaling. FIGS. 8C-FIG. 8E are a set of bar plots quantifying the amounts of tonic signaling in VH-only anti-BCMA CAR cell clones.

FIGS. 9A-9B are a set of schematics, bar, and Kaplan-Meier plots that show VH-only anti-BCMA CAR cells are effective in vivo. FIG. 9A is a schematic illustrating the injection mouse tumor model and bar plot that shows the total bioluminescent tumor imaging (BLI) of each treatment group after tumor injection. FIG. 9B is a Kaplan-Meier plot that shows mouse survival after tumor injection.

FIGS. 10A-10D are a set of microarrays that show VH-only anti-BCMA binder specificity. FIG. 10A is a cell microarray that shows binding of a VH-only anti-BCMA binding test antibody to select human proteins. FIG. 10B is a cell microarray that shows binding of a negative control antibody to select human proteins. FIG. 10C is a cell microarray that shows binding of Rituximab biosimilar to select human proteins. FIG. 10D is a cell microarray that shows binding of a PBS, secondary antibody negative control to select human proteins.

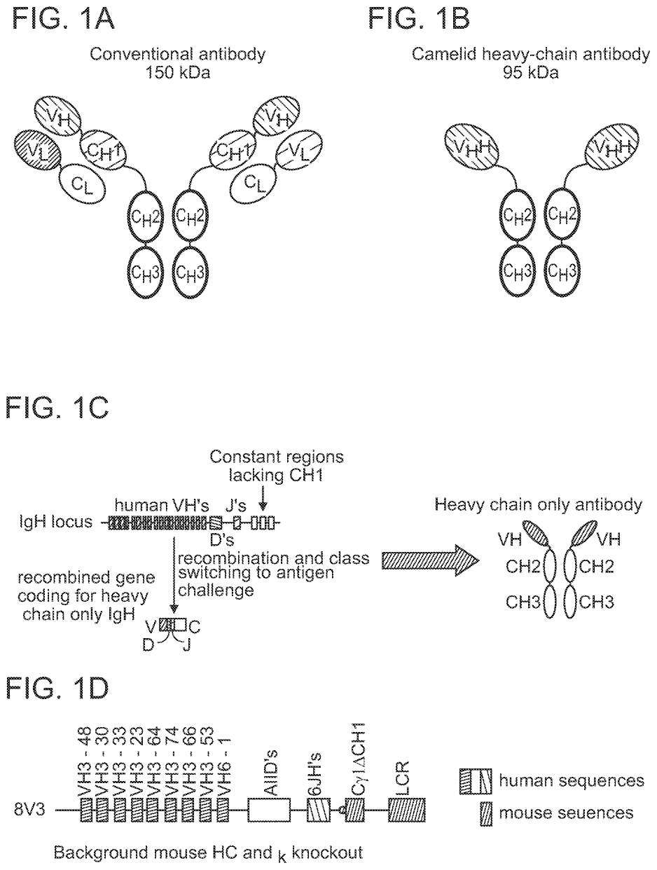

### DETAILED DESCRIPTION OF THE DISCLOSURE

### Definitions

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present disclosure.

As used in the description and the appended claims, the singular forms “a”, “an”, and “the” mean “one or more” and therefore include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about.”

The term “approximately” as used herein refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The transitional term “comprising,” which is synonymous with “include(s)”, “including,” “contain(s)”, “containing,” or “characterized by,” is inclusive or open-ended and does not exclude additional, unrecited elements or method steps. By contrast, the transitional phrases “consist(s) of” and “consisting of” excludes any element or method step not specified in the claim (or the specific element or method step with which the phrase “consisting of” is associated). The transitional phrase “consisting essentially of” limits the scope of a claim to the specified elements and method or steps and “unrecited elements and method steps that do not materially affect the basic and novel characteristic(s)” of the claimed disclosure.

### Nucleic Acids

In one aspect, the disclosure provides nucleic acids that encode a chimeric antigen receptor (CAR) made up of an extracellular domain that contains a first antigen recognition domain made up of solely a fully human, single variable heavy (VH) domain that binds to a B-cell maturation antigen (BCMA) epitope, a transmembrane domain, and an intracellular signaling domain that contains a singling domain. Because the antigen recognition domain “consists of” a VH, it contains none of a VL domain, a CL domain, or a CH1 domain.

As known in the art, the term “nucleic acid” as used herein refers to a polymer of nucleotides, each of which are organic molecules consisting of a nucleoside (a nucleobase and a five-carbon sugar) and a phosphate. The term nucleotide, unless specifically stated or obvious from context, includes nucleosides that have a ribose sugar (i.e., a ribonucleotide that forms ribonucleic acid, RNA) or a 2′-deoxyribose sugar (i.e., a deoxyribonucleotide that forms deoxyribonucleic acid, DNA). Nucleotides serve as the monomeric units of nucleic acid polymers or polynucleotides. The four nucleobases in DNA are guanine (G), adenine (A), cytosine (C) and thymine (T). The four nucleobases in RNA are guanine (G), adenine (A), cytosine (C) and uracil (U). Nucleic acids are linear chains of nucleotides (e.g., at least 3 nucleotides) chemically bonded by a series of ester linkages between the phosphoryl group of one nucleotide and the hydroxyl group of the sugar (i.e., ribose or 2′-deoxyribose) in the adjacent nucleotide. In the present context, it is understood that the nucleic acids are exogenous to the cells into which they may be introduced.

Fully human, heavy chain-only antibodies are known in the art. See, Roovers et al., Cancer Immunol. Immunother. 56(3):303-317 (2007), Tang et al., Mol. Cancer Ther. 12:416-26 (2013), Sanchez-Martin et al., Proc. Natl. Acad. Sci. USA 110(34):13791-6 (2013), Rouet et al., J. Biol. Chem. 290:11905-17 (2015), Ingram et al., Proc. Natl. Acad. Sci. USA 115(15):3912-3917 (2018), Zhu et al., Protein Expr. Purif. 157:57-62 (2019), and Wang et al., Antibodies (Basel) 8(1):25 (2019).

BCMA, also known as tumor necrosis factor receptor superfamily member 17 (TNFRSF17), is a cell surface receptor that recognizes B-cell activating factor (BAFF).

BCMA is predominantly expressed in mature B lymphocytes. BCMA has been used as a CAR target in clinical trials; however, these therapies have suffered from a number of setbacks, such as for example, short CAR cell persistence of less than 6 months (Brudno et al., J. Clin. Oncol. 36:2267-2280 (2018), Raje et al., N. Engl. J. Med. 380:1726-1737 (2019), Berdeja et al., Lancet 398:314-324 (2021)).

VH domains derived from a heavy chain-only antibodies may be subdivided into seven interconnected regions (or motifs), four heavy chain framework regions (FWRs) and three heavy chain complementarity-determining regions (CDRs). The FWRs have conserved sequences and make up about 85% of the variable region of an antibody. The FWRs form scaffolds for the CDRs and maintain the overall structure of the variable region. The CDRs have highly variable sequences and are responsible for the antibody's specific binding to the antigen's epitope.

The fully human VH-only BCMA recognition domains contain the heavy framework regions FWR1, FWR2, FWR3 and FWR4, and the heavy chain complementarity-determining regions CDR1, CDR2, and CDR3.

In some embodiments, the antigen recognition domain has the amino acid sequence set forth below (SEQ ID NO: 1) (derived from a VH domain referred to herein as PR000940), which includes from 5′ to 3′, all 7 motifs (or “regions”) set forth in Table 1:

```
    1 evqllesggg lvqpggslrl scaasgftfs
        syamiwvrqa pgkglewvsg isesggstyy
  61 adsvkgrfti srdnskntly lqmnslraed
        tavyfcvkdl ddiltgyykd ywgqgtlvtv
121 ss
```

In some embodiments, the antigen recognition domain has the nucleic acid sequence codon optimized set forth below (SEQ ID NO: 88), encoding, from 5′ to 3′, SEQ ID NO: 1

```
    1 gaggtgcagc tgctggagag cggcggcggc
        ctggtgcagc ccggcggcag cctgaggctg
  61 agctgcgccg ccagcggctt caccttcagc
        agctacgcca tgatctgggt gaggcaggcc
121 cccggcaagg gcctggagtg ggtgagcggc
        atcagcgaga gcggcggcag cacctactac
181 gccgacagcg tgaagggcag gttcaccatc
        agcagggaca acagcaagaa caccctgtac
241 ctgcagatga acagcctgag ggccgaggac
        accgccgtgt acttctgcgt gaaggacctg
301 gacgacatcc tgaccggcta ctacaaggac
        tactggggcc agggcaccct ggtgaccgtg
361 agcagc.
```

TABLE 1
Sequences of PR000940 regions

| Region | Amino acid sequence |
|---|---|
| HFWR1 (SEQ ID NO: 2) | EVQLLESGGGLVQP |
| | GGSLRLSCAAS |
| HCDR1 (SEQ ID NO: 3) | GFTFSSY |
| HFWR2 (SEQ ID NO: 4) | AMIWVRQAPGKGLE |
| | WVSGI |
| HCDR2 (SEQ ID NO: 5) | SESGGS |
| HFWR3 (SEQ ID NO: 6) | TYYADSVKGRFTISR |
| | DNSKNTLYLQMNSLR |
| | AEDTAVYFCVK |
| HCDR3 (SEQ ID NO: 7) | DLDDILTGYYKDY |
| HFWR4 (SEQ ID NO: 8) | WGQGTLVTVSS |

In some embodiments, the antigen recognition domain has the amino acid sequence set forth below (SEQ ID NO: 9) (derived from a VH domain referred to herein PR000943), which includes from 5′ to 3′, all 7 motifs set forth in Table 2:

```
  1 evqlvetggg liqpggslrl scaasgfpvs
      snymswvrqa pgkglewvsv ifsggrtyya
61 dsvkgrftis rdnskntlyl qmnslraedt
      avyycarrnw idgrdafdiw gqgtmvtvss
```

In some embodiments, the antigen recognition domain has the nucleic acid sequence codon optimized set forth below (SEQ ID NO: 89), encoding, from 5′ to 3′, SEQ ID NO: 9.

| | |
|---|---|
| 1 | gaggtgcagc tggtggagac cggcggcggc ctgatccagc ccggcggcag cctgaggctg |
| 61 | agctgcgccg ccagcggctt ccccgtgagc agcaactaca tgagctgggt gaggcaggcc |
| 121 | cccggcaagg gcctggagtg ggtgagcgtg atcttcagcg gcggcaggac ctactacgcc |
| 181 | gacagcgtga agggcaggtt caccatcagc agggacaaca gcaagaacac cctgtacctg |
| 241 | cagatgaaca gcctgagggc cgaggacacc gccgtgtact actgcgccag gaggaactgg |
| 301 | atcgacggca gggacgcctt cgacatctgg ggccagggca ccatggtgac cgtgagcagc |

TABLE 2
Sequences of PR000943 regions

| Region | Amino acid sequence |
|---|---|
| FWR1 (SEQ ID NO: 10) | EVQLVETGGGLIQPGGSLR |
| | LSCAAS |
| CDR1 (SEQ ID NO: 11) | GFPVSSN |
| FWR2 (SEQ ID NO: 12) | YMSWVRQAPGKGLEWVSVI |
| CDR2 (SEQ ID NO: 13) | FSGGR |
| FWR3 (SEQ ID NO: 14) | TYYADSVKGRFTISRDNSK |
| | NTLYLQMNSLRAEDTAVYY |
| | CAR |
| CDR3 (SEQ ID NO: 15) | RNWIDGRDAFDI |
| FWR4 (SEQ ID NO: 16) | WGQGTMVTVSS |

In some embodiments, the antigen recognition domain has the amino acid sequence set forth below (SEQ ID NO: 17) (derived from a VH domain referred to herein as PR001035), which includes from 5′ to 3′, all 7 motifs set forth in Table 3:

| | |
|---|---|
| 1 | evqlvetggg liqpggslrl scaasgftvs snymtwvrqa pgkglewvsv ifsggstyya |
| 61 | dsvkgrftis rdnskntlyl qmnslraedt avyycarria vaghdvfdiw gqgtmvtvss |

In some embodiments, the antigen recognition domain has the nucleic acid sequence codon optimized set forth below (SEQ ID NO: 90), encoding, from 5′ to 3′, SEQ ID NO: 17.

| | |
|---|---|
| 1 | gaggtgcagc tggtggagac cggcggcggc ctgatccagc ccggcggcag cctgaggctg |
| 61 | agctgcgccg ccagcggctt caccgtgagc agcaactaca tgacctgggt gaggcaggcc |
| 121 | cccggcaagg gcctggagtg ggtgagcgtg atcttcagcg gcggcagcac ctactacgcc |
| 181 | gacagcgtga agggcaggtt caccatcagc agggacaaca gcaagaacac cctgtacctg |
| 241 | cagatgaaca gcctgagggc cgaggacacc gccgtgtact actgcgccag gaggatcgcc |
| 301 | gtggccggcc acgacgtgtt cgacatctgg ggccagggca ccatggtgac cgtgagcagc |

TABLE 3
Sequences of PR001035 regions

| Region | Amino acid sequence |
|---|---|
| FWR1 (SEQ ID NO: 18) | EVQLVETGGGLIQPGGSLR |
| | LSCAAS |
| CDR1 (SEQ ID NO: 19) | GFTVSSN |
| FWR2 (SEQ ID NO: 20) | YMTWVRQAPGKGLEWVSVI |
| CDR2 (SEQ ID NO: 21) | FSGGS |
| FWR3 (SEQ ID NO: 22) | TYYADSVKGRFTISRDNSK |
| | NTLYLQMNSLRAEDTAVYY |
| | CAR |
| CDR3 (SEQ ID NO: 23) | RIAVAGHDVFDI |
| FWR4 (SEQ ID NO: 24) | WGQGTMVTVSS |

In some embodiments, the antigen recognition domain has the amino acid sequence set forth below (SEQ ID NO: 25) (derived from a VH domain referred to herein as PR001046), which includes from 5′ to 3′ all 7 motifs set forth in Table 4:

| | |
|---|---|
| 1 | evqlvetggg liqpggslrl scaasgftvs dnymtwvrqa pgkglewvsv ifsggntyya |
| 61 | dsvkgrftis rdnakntlyl qmnslraedt alyycarrny ddtrgtdvfd iwgqgtmvtv |
| 121 | ss |

In some embodiments, the antigen recognition domain has the nucleic acid sequence codon optimized set forth below (SEQ ID NO: 91), encoding, from 5′ to 3′, SEQ ID NO: 25.

| | |
|---|---|
| 1 | gaggtgcagc tggtggagac cggcggcggc ctgatccagc ccggcggcag cctgaggctg |
| 61 | agctgcgccg ccagcggctt caccgtgagc agcaactaca tgacctgggt gaggcaggcc |
| 121 | cccggcaagg gcctggagtg ggtgagcgtg atcttcagcg gcggcagcac ctactacgcc |
| 181 | gacagcgtga agggcaggtt caccatcagc agggacaaca gcaagaacac cctgtacctg |
| 241 | cagatgaaca gcctgagggc cgaggacacc gccgtgtact actgcgccag gaggatcgcc |
| 301 | gtggccggcc acgacgtgtt cgacatctgg ggccagggca ccatggtgac cgtgagcagc |

TABLE 4
Sequences of PR001046 regions

| Region | Amino acid sequence |
|---|---|
| FWR1 (SEQ ID NO: 26) | EVQLVETGGGLIQPGGSLR |
| | LSCAAS |
| CDR1 (SEQ ID NO: 27) | GFTVSDN |
| FWR2 (SEQ ID NO: 28) | YMTWVRQAPGKGLEWVSVI |
| CDR2 (SEQ ID NO: 29) | FSGGN |
| FWR3 (SEQ ID NO: 30) | TYYADSVKGRFTISRDNAK |
| | NTLYLQMNSLRAEDTALYY |
| | CAR |
| CDR3 (SEQ ID NO: 31) | RNYDDTRGTDVFDI |
| FWR4 (SEQ ID NO: 32) | WGQGTMVTVSS |

In some embodiments, the antigen recognition domain is a variant of any one of (and therefore differs from) the BCMA recognition domains of SEQ ID NOs: 1, 9, 17, and 25. In some embodiments, the variant of any one of SEQ ID NOs: 1, 9, 17, and 25 retains at least about 95% identity with the corresponding non-variant SEQ ID NOs: 1, 9, 17, and 25, respectively. In some embodiments, the variant of any one of SEQ ID NOs: 1, 9, 17, and 25 retains at least about 98%, e.g., 99% identity with the corresponding non-variant SEQ ID NOs: 1, 9, 17, and 25, respectively. The term “identity” refers to the percentage of residues that are identical in the two sequences when the sequences are optimally aligned. If, in the optimal alignment, a position in a first sequence is occupied by the same amino acid as the corresponding position in the second sequence, the sequences exhibit identity with respect to that position. The percentage of identity determines the number of identical residues over a defined length in a given alignment. Thus, the level of identity between two sequences or (“percent sequence identity”) is measured as a ratio of the number of identical positions shared by the sequences with respect to the number of positions compared (i.e., percent sequence identity=(number of identical positions/total number of positions compared)×100). A gap, i.e., a position in an alignment where a residue is present in one sequence but not in the other, is regarded as a position with non-identical residues and is counted as a compared position.

Variants are well understood to those of skill in the art. In the context of the present fully human VH-only BCMA binding domains, they can involve amino acid sequence modifications that typically fall into one or more of three classes: substitutional, insertional, or deletional variants. In the present disclosure, the variants are “functional” in the sense that they retain binding to BCMA.

The term “substitutional variant” refers to at least one amino acid in a native or starting sequence of a polypeptide is removed and a different amino acid is inserted in its place. The substitutions may be single, where only one amino acid in the polypeptide molecule is substituted, or they may be multiple, where two or more amino acids are substituted in the same polypeptide molecule.

In some embodiments, the variant includes one of more amino acid substitutions that are conservative in nature. The term “conservative substitution” refers to at least one amino acid in a native or starting sequence of a polypeptide is substituted with a different amino acid of similar size, charge, or polarity. Examples of conservative substitutions include the substitution of a non-polar (hydrophobic) residue for another non-polar residue, for example exchanging isoleucines, valines or leucines. Likewise, examples of conservative substitutions include the substitution of one polar (hydrophilic) residue for another, for example exchanging between arginine and lysine, between glutamine and asparagine, and between glycine and serine. Additional examples of conservative substitutions include that of a basic amino acid such as lysine, arginine or histidine for another, or the substitution of one acidic residue such as aspartic acid or glutamic acid for another acidic residue. Further examples of conservative substitutions include the substitution of amino acid residues with similar chemical properties, for example, exchanging between serine with a threonine, where each residue has a hydroxy group (—OH).

The term “insertional variants” refers to variants with one or more amino acids inserted immediately adjacent to an amino acid at a particular position in a native or starting sequence. The term “immediately adjacent” to an amino acid as used herein refers to an amino acid connection through either the α-carboxy or α-amino functional group of the amino acid.

The term “deletional variants” as used herein refers to variants with one or more amino acids in the native or starting amino acid sequence removed. Ordinarily, deletional variants will have one or more amino acids deleted in a particular region of the molecule.

In some embodiments, a variant of an antigen recognition domain having an amino acid sequence of any one of SEQ ID NOs: 1, 9, 17, and 25 may have one or more amino acid substitutions. Representative amino acid substitutions in SEQ ID NOs: 1, 9, 17, and 25 are exemplified in the following alignment with downward carets (“v”) and bolded amino acid residues for substitutional variants and dashes for deletional variants or insertional variants:

| | v v v vv vvv v | |
|---|---|---|
| 1 | evqllesggg lvqpggslrl scaasgftfs syamiwvrqa | (SEQ ID NO: 1) |
| 1 | evqlvetggg liqpggslrl scaasgfpvs snymswvrqa | (SEQ ID NO: 9) |
| 1 | evqlvetggg liqpggslrl scaasgftvs snymtwvrqa | (SEQ ID NO: 17) |
| 1 | evqlvetggg liqpggslrl scaasgftvs dnymtwvrqa | (SEQ ID NO: 25) |
| | v v v v | |
| 41 | pgkglewvsg isesggstyy adsvkgrfti srdnskntly | (SEQ ID NO: 1) |
| 41 | pgkglewvsv if-sggrtyy adsvkgrfti srdnskntly | (SEQ ID NO: 9) |
| 41 | pgkglewvsv if-sggstyy adsvkgrfti srdnskntly | (SEQ ID NO: 17) |
| 41 | pgkglewvsv if-sggntyy adsvkgrfti srdnakntly | (SEQ ID NO: 25) |
| | v v vvvv vvvvv v vv v v | |
| 81 | lqmnslraed tavyfcvkdl ddiltgy-yk dywgqgtlvt vss | (SEQ ID NO: 1) |
| 80 | lqmnslraed tavyycarrn wid-grdaf diwgqgtmvt vss | (SEQ ID NO: 9) |
| 80 | lqmnslraed tavyycarri ava-ghdvf diwgqgtmvt vss | (SEQ ID NO: 17) |
| 81 | lqmnslraed talyycarrn yddtrgtdvf diwgqgtmvt vss | (SEQ ID NO: 25) |

In some embodiments, the substitutional variant is SEQ ID NO: 1 with a I35S substitution. In some embodiments, the substitutional variant is SEQ ID NO: 9 with a S31D substitution. In some embodiments, a substitutional variant is SEQ ID NO: 17 with a S75A substitution. In some embodiments, SEQ ID NO: 25 with a A74S substitution. In some embodiments, the deletional variant is SEQ ID NO: 1 with a deletion of the glutamic acid (E) at position 53 (E53del). In some embodiments, the substitutional variant is SEQ ID NO: 9 with an aspartic acid (D) deletion at position 105 (D105del). In some embodiments, the substitutional variant is SEQ ID NO: 17 with an aspartic acid (D) deletion at position 105 (D105del). In some embodiments, a deletional variant is SEQ ID NO: 25 with a threonine (T) and arginine I deletion at positions 104 to 105 (T104_L105del). In some embodiments, the insertion variant is SEQ ID NO: 9 with a glutamic acid (E) inserted between phenylalanine (F) at position 52 and serine (S) at position 53 (F52_S53insE). In some embodiments, the substitutional variant is SEQ ID NO: 17 with a leucine (L) and an arginine (R) inserted between alanine (A) at position 102 and glycine (G) at position 103 (A102_G103insLR). In some embodiments, the substitutional variant is SEQ ID NO: 9 with a leucine (L) inserted between aspartic acid (D) at position 102 and glycine (G) at position 103. In some embodiments, the substitutional variant is SEQ ID NO: 17 with an arginine (R) inserted between alanine (A) at position 102 and glycine (G) at position 103.

In some embodiments, the extracellular domain includes a second antigen recognition domain also solely made up (i.e., consisting of) of a fully human, single VH domain that binds to a second BCMA epitope. The BCMA epitopes to which the first and second antigen recognition domains bind may be the same epitope or different epitopes.

In some embodiments, the first and second antigen recognition domains are connected by a linker. In some embodiments, the linker has an amino acid sequence of GGGX (SEQ ID NO: 33), GGGGX (SEQ ID NO: 34), or GSSGSX (SEQ ID NO: 35), where X is either cysteine (C) or serine (S). In some embodiments, the linker has a repeating sequence of SEQ ID NOs 33-35. In some embodiments, the linker has an amino acid sequence of GGGGSGGGGSGGGGSGGGGS (SEQ ID NO: 36), GSTSGSGKPGSGEGSTKG (SEQ ID NO: 37), KESGSVSSEQLAQFRSLD (SEQ ID NO: 38), EGKSSGSGSESKST (SEQ ID NO: 39), or GSAGSAAGSGEF (SEQ ID NO: 40).

The antigen recognition domain may be derived from a heavy chain-only, fully human antibody in accordance with known procedures. To be “fully human”, the entire sequence of the antigen recognition domain (e.g., an antibody or antibody fragment from which the antigen recognition domain originates) is derived from the human variable gene segments, diversity gene segments, and joining gene segments. Such fully human antigen recognition domains derived from antibodies may be produced in a transgenic, non-human mammal which has had its endogenous antibody heavy chain locus and light chain locus knocked out or inactivated (thus making it unable to produce endogenous antibodies) and providing one or more human heavy chain loci into that mammal. Such a transgenic non-human mammal having a synthetic arrangement of only a human heavy chain may produce single-domain binding antibodies that bind an antigen without a light chain. See, U.S. Pat. No. 10,638,735 and U.S. Patent Application Publication 2023/0322953. In some embodiments, the antigen recognition domain is derived from heavy chain-only antibodies generated from such transgenic mice. The antigen recognition domains derived from heavy chain-only antibodies would not include any light chain domains (i.e., a VL domain or a VH domain).

The transmembrane domain of the CAR connects the extracellular domain (that includes the antigen recognition domain) to the intracellular signaling region. In some embodiments, the transmembrane domain is directly connected to the extracellular domain.

In some embodiments, the transmembrane domain is derived from CD3a, CD30, CD3γ, CD3ζ, CD3ε, CD4, CD5, CD8α, CD9, CD16, CD22, CD28, CD33, CD37, CD45, CD64, CD80, CD86, CD134, CD137 (4-BB or TNF Receptor Superfamily Member 9 (TNFRSF9)), CD154, FcεRIα, FcεRIβ, FcεRI1, ICOS, KIR2DS2, MHC class I, MHC class II, or NKG2D. In some embodiments, the transmembrane domain is derived from CD3ζ, CD4, CD8a, CD28, or CD137, representative sequences of which are listed in Table 5.

TABLE 5
Transmembrane domain sequences

| Transmembrane domain | Sequence |
|---|---|
| CD3ζ (SEQ ID NO: 41) | LCYLLDGILFIYGVILTALFL |
| CD4 (SEQ ID NO: 42) | MALIVLGGVAGLLLFIGLGIFF |
| CD8α (SEQ ID NO: 43) | IYIWAPLAGTCGVLLLSLVITLYC |
| CD28 (SEQ ID NO: 44) | FWVLVVVGGVLACYSLLVTVAFIIFWV |
| CD137 (SEQ ID NO: 45) | IISFFLALTSTALLFLLFFLTLRFSVV |

The amino acid sequence of a naturally occurring transmembrane domain may be modified by an amino acid substitution to avoid binding of such regions to the transmembrane domain of the same or different surface membrane proteins to minimize interactions with other members of a receptor complex. See, e.g., U.S. Patent Application Publication 2021/0101954; Soudais et al., Nat Genet 3:77-81 (1993); Muller et al., Front. Immunol. 12:639818-13 (2021); and Elazar et al., eLife 11:e75660-29 (2022).

In some embodiments, the CAR further includes a hinge domain disposed between the antigen recognition domain and the transmembrane domain. A hinge domain may provide flexibility in terms of allowing the antigen recognition domain to obtain an optimal orientation for antigen-binding, thereby enhancing antitumor activities of the cell expressing the CAR.

In some embodiments, the hinge domain is derived from IgA, IgD, IgE, IgG, or IgM. In some embodiments, the hinge domain is derived from CD3ζ, CD4, CD8a, CD28, IgG1, IgG2, or IgG4, representative sequences of which are listed in Table 6.

TABLE 6
Hinge domain sequences

| Hinge domain | Sequence |
|---|---|
| CD3ζ (SEQ ID NO: 46) | QSFGLLDPK |
| CD4 (SEQ ID NO: 47) | LSEGDKVKMDSRIQVLSRGVNQT |
| CD8α (SEQ ID NO: 48) | KPTTTPAPRPPTPAPTIASQPLS |
| | KRPEACRPAAGGAVHTRGLDFAC |
| | DIY |
| CD28 (SEQ ID NO: 49) | IEVMYPPPYLDNERSNGTIIHVK |
| | GKHLCPSPLFPGPSKP |
| IgG1 (SEQ ID NO: 50) | EPKSCDKTHTCPPCPAPELLGG |
| IgG2 (SEQ ID NO: 51) | ERKCCVECPPCPAPPAAA |
| IgG4 (SEQ ID NO: 52) | ESKYGPPCPPCPAPEFLGG |

The intracellular domain contains a signaling domain that enables intracellular signaling and immune cell function. The signaling domain may include a primary signaling domain and/or a co-stimulatory signaling domain. In some embodiments, the intracellular domain includes one or more phosphorylatable intracellular motifs (ITAMs) capable of delivering an immune activating signal. In some embodiments, the intracellular domain is capable of delivering a signal approximating that of natural ligation of an ITAM-containing molecule or receptor complex such as a TCR receptor complex.

In some embodiments, the intracellular signaling domain includes a plurality, e.g., 2 or 3, costimulatory signaling domains described herein, e.g., selected from 4-1BB, CD28, CD27, ICOS, and OX40. In some embodiments, the intracellular signaling domain may include a CD3ζ domain as a primary signaling domain, and any of the following pairs of costimulatory signaling domains from the extracellular to the intracellular direction: 4-11B1-CD27; CD27-4-1BB; 4-1BB-CD28; CD28-4-1BB; OX40-CD28; CD28-OX40; CD28-4-1BB and 4-1BB-CD28. In some embodiments the primary signaling domain is derived from CD3ζ, CD27, CD28, CD40, KIR2DS2, MyD88, OX40. In some embodiments, the co-stimulatory signaling domain is derived from one or more of CD3γ, CD3δ, CD3ε, CD3ζ, CD27, CD40, CD28, CD72, CD80, CD86, CLEC-1, 4-11B1, TYROBP (DAP12), Dectin-1, Fca˜RI, FcγRI, FcγRII, FcεRI, FcεRII, IL-2RB, ICOS, KIR2DS2, MyD88, OX40, and ZAP70. Representative sequences of signaling domains are listed in Table 7.

TABLE 7
Signaling domain sequences

| Signaling | |
|---|---|
| domain | Sequence |
| CD3ζ | RVKFSRSADAPAYKQGQNQLYNELNL |
| (SEQ ID NO: 53) | GRREEYDVLDKRRGRDPEMGGKPRRK |
| | NPQEGLYNELQKDKMAEAYSEIGMKG |
| | ERRRGKGHDGLYQGLSTATKDTYDAL |
| | HMQALPPR |
| CD3ζ variant | RVKFSRSADAPAYQQGQNQLYNELNL |
| (SEQ ID NO: 54) | GRREEYDVLDKRRGRDPEMGGKPRRK |
| | NPQEGLYNELQKDKMAEAYSEIGMKG |
| | ERRRGKGHDGLYQGLSTATKDTYDAL |
| | HMQALPPR |
| CD3ϵ | KNRKAKAKPVTRGAGAGGRQRGQNKE |
| (SEQ ID NO: 55) | RPPPVPNPDYEPIRKGQRDLYSGLNQ |
| | RRI |
| CD4 | RSKRSRLLHSDYMNMTPRRPGPTRKH |
| (SEQ ID NO: 56) | YQPYAPPRDFAAYRS |
| CD27 | QRRKYRSNKGESPVEPAEPCHYSCPR |
| (SEQ ID NO: 57) | EEEGSTIPIQEDYRKPEPACSP |
| CD28 | TYCFAPRCRERRRNERLRRESVRPV |
| (SEQ ID NO: 58) | |
| CD40 | MIETYNQTSPRSAATGLPISMK |
| (SEQ ID NO: 59) | |
| CD80 | TYCFAPRCRERRRNERLRRESVRPV |
| (SEQ ID NO: 60) | |
| CD86 | KWKKKKRPRNSYKCGTNTMEREESEQ |
| (SEQ ID NO: 61) | TKKREKIHIPERSDEAQRVFKSSKTS |
| | SCDKSDTCF |
| CD137 | KRGRKKLLYIFKQPFMRPVQTTQEED |
| (SEQ ID NO: 62) | GCSCRFPEEEEGGCELLCARPRRSPA |
| | QEDGKVYINMPGRG |
| DAP10 | CWLTKKKYSSSVHDPNGEYMFMRAVN |
| (SEQ ID NO: 63) | TAKKSRLTDVTL |
| DAP12 | YFLGRLVPRGRGAAEAATRKQRITET |
| (SEQ ID NO: 64) | ESPYQELQGQRSDVYSDLNTQRPYYK |
| FcϵRI | RLKIQVRKAAITSYEKSDGVYTGLST |
| (SEQ ID NO: 65) | RNQETYETLKHEKPPQ |
| ICOS | RSKRSRLLHSDYMNMTPRRPGPTRKH |
| (SEQ ID NO: 66) | YQPYAPPRDFAAYRS |
| KIR2DS2 | HRWCSNKKNAAVMDQEPAGNRTVNSE |
| (SEQ ID NO: 67) | DSDEQDHQEVSYA |
| MyD88 | MAAGGPGAGSAAPVSSTSSLPLAALN |
| (SEQ ID NO: 68) | MRVRRRLSLFLNVRTQVAADWTALAE |
| | EMDFEYLEIRQLETQADPTGRLLDAW |
| | QGRPGASVGRLLELLTKLGRDDVLLE |
| | LGPSIEEDCQKYILKQQQEEAEKPLQ |
| | VAAVDSSVPRTAELAGITTLDDPLGH |
| | MPERFDAFICYCPSDIQFVQEMIRQL |
| | EQTNYRLKLCVSDRDVLPGTCVWSIA |
| | SELIEKRCRRMVVVVSDDYLQSKECD |
| | FQTKFALSLSPGAHQKRLIPIKYKAM |
| | KKEFPSILRFITVCDYTNPCTKSWFW |
| | TRLAKALSLP |
| OX40 | ALYLLRRDQRLPPDAHKPPGGGSFRT |
| (SEQ ID NO: 69) | PIQEEQADAHSTLAKI |
| ZAP70 | MPDPAAHLPFFYGSISRAEAEEHLKL |
| (SEQ ID NO: 70) | AGMADGLFLLRQCLRSLGGYVLSLVH |
| | DVRFHHFPIERQLNGTYAIAGGKAHC |
| | GPAELCEFYSRDPDGLPCNLRKPCNR |
| | PSGLEPQPGVFDCLRDAMVRDYVRQT |
| | WKLEGEALEQAIISQAPQVEKLIATT |
| | AHERMPWYHSSLTREEAERKLYSGAQ |
| | TDGKFLLRPRKEQGTYALSLIYGKTV |
| | YHYLISQDKAGKYCIPEGTKFDTLWQ |
| | LVEYLKLKADGLIYCLKEACPNSSAS |
| | NASGAAAPTLPAHPSTLTHPQRRIDT |
| | LNSDGYTPEPARITSPDKPRPMPMDT |
| | SVYESPYSDPEELKDKKLFLKRDNLL |
| | IADIELGCGNFGSVRQGVYRMRKKQI |
| | DVAIKVLKQGTEKADTEEMMREAQIM |
| | HQLDNPYIVRLIGVCQAEALMLVMEM |
| | AGGGPLHKFLVGKREEIPVSNVAELL |
| | HQVSMGMKYLEEKNFVHRDLAARNVL |
| | LVNRHYAKISDFGLSKALGADDSYYT |
| | ARSAGKWPLKWYAPECINFRKFSSRS |
| | DVWSYGVTMWEALSYGQKPYKKMKGP |
| | EVMAFIEQGKRMECPPECPPELYALM |
| | SDCWIYKWEDRPDFLTVEQRMRACYY |
| | SLASKVEGPPGSTQKAEAACA |

In some embodiments, the signaling domain is derived from CD3ζ and the co-stimulatory domain is derived from 4-11B1. In some embodiments, the stimulatory domain is derived from CD3ζ and the co-stimulatory domain is derived from CD28. In some embodiments, the stimulatory domain is derived from CD3ζ and the co-stimulatory domain is derived from 4-11B1 and CD28.

Sequences of 4-11B1 and CD28 are provided in Table 7 and additional isoforms of CD28 are provided in Table 8.

TABLE 8
CD28 isoform sequences

| Isoform | Sequence |
|---|---|
| CD28 isoform 1 | MLRLLLALNLFPSIQVTGNKILVKQSPMLV |
| NP_006130.1 | AYDNAVNLSCKYSYNLFSREFRASLHKGLD |
| (SEQ ID NO: 71) | SAVEVCVVYGNYSQQLQVYSKTGFNCDGKL |
| | GNESVTFYLQNLYVNQTDIYFCKIEVMYPP |
| | PYLDNEKSNGTIIHVKGKHLCPSPLFPGPS |
| | KPFWVLVVVGGVLACYSLLVTVAFIIFWVR |
| | SKRSRLLHSDYMNMTPRRPGPTRKHYQPYA |
| | PPRDFAAYRS |
| CD28 isoform 2 | MLRLLLALNLFPSIQVTGNKILVKQSPMLV |
| NP_001230006.1 | AYDNAVNLSWKHLCPSPLFPGPSKPFWVLV |
| (SEQ ID NO: 72) | VVGGVLACYSLLVTVAFIIFWVRSKRSRLL |
| | HSDYMNMTPRRPGPTRKHYQPYAPPRDFAA |
| | YRS |
| CD28 isoform 3 | MLRLLLALNLFPSIQVTGKHLCPSPLFPGP |
| NP_001230007.1 | SKPFWVLVVVGGVLACYSLLVTVAFIIFWV |
| (SEQ ID NO: 73) | RSKRSRLLHSDYMNMTPRRPGPTRKHYQPY |
| | APPRDFAAYRS |
| CD28 isoform 4 | MPCGLSALIMCPKGMVAVVVAVDDGDSQAL |
| NP_001397910.1 | AGNKILVKQSPMLVAYDNAVNLSCKYSYNL |
| (SEQ ID NO: 74) | FSREFRASLHKGLDSAVEVCVVYGNYSQQL |
| | QVYSKTGFNCDGKLGNESVTFYLQNLYVNQ |
| | TDIYFCKIEVMYPPPYLDNEKSNGTIIHVK |
| | GKHLCPSPLFPGPSKPFWVLVVVGGVLACY |
| | SLLVTVAFIIFWVRSKRSRLLHSDYMNMTP |
| | RRPGPTRKHYQPYAPPRDFAAYRS |

In some embodiments, the CAR further includes a signal peptide. The term “signal peptide” as used herein refers to a short (e.g., 5-30 or 10-100 amino acids long) stretch of amino acids typically at the N-terminus of a protein that directs the transport of the protein. The signal peptide allows for the association of the mRNA and ribosome with the endoplasmic reticulum, insertion of the newly translated protein into the translocon, translocation, and trafficking of the protein product to the plasma membrane. Often, the signal peptide is cleaved off during the post-translational modification of a protein by a cell. The signal peptide may be evaluated in silico to ensure efficient functional cleavage.

In some embodiments, the signal peptide is derived from the albumin, CD8a, CD33, erythropoietin, IL-2, human or mouse Ig-kappa chain V-III (IgK VIII), tissue plasminogen activator (tPA), secreted alkaline phosphatase (SEAP), as well as synthetic sequences. Representative sequences of signal peptides are listed in Table 9.

TABLE 9
Signal peptide sequences

| Signal peptide | Sequence |
|---|---|
| Albumin (SEQ ID NO: 75) | MKWVTFISLLFLFSSAYS |
| Synthetic, modified | MKWVTFISLLFLFSSSSRA |
| albumin (SEQ ID NO: 76) | |
| CD8α (SEQ ID NO: 77) | MALPVTALLLPLALLLHAARP |
| CD33 (SEQ ID NO: 78) | MPLLLLLPLLWAGALA |
| Erythropoietin (EPO) | MGVHECPAWLWLLLSLLSLPL |
| (SEQ ID NO: 79) | GLPVLG |
| IL-2 (SEQ ID NO: 80) | MYRMQLLSCIALSLALVTNS |
| Mouse IgK VIII | METDTLLLWVLLLWVPGSTG |
| (SEQ ID NO: 81) | |
| Human IgK VIII | MEAPAQLLFLLLLWLPDTTG |
| (SEQ ID NO: 82) | |
| Human IgK V-IV | MVLQTQVFISLLLWISGAYG |
| (SEQ ID NO: 83) | |
| Synthetic, modified human | MEAPAQLLFLLLLWLPSSRA |
| IgK VIII (SEQ ID NO: 84) | |
| tPA (SEQ ID NO: 85) | MDAMKRGLCCVLLLCGAVFVS |
| | PS |
| SEAP (SEQ ID NO: 86) | MLLLLLLLGLRLQLSLG |
| Consensus (SEQ ID NO: 87) | MLLLLLLLLLLALALA |
| Synthetic secrecon | MWWRLWWLLLLLLLLWPMVWA |
| (SEQ ID NO: 88) | |

In some embodiments, the sequence of the nucleic acid encoding the CAR may be codon-optimized to match balance frequency of codon usage in the organism and cell type (e.g., a human immune cell) expressing the CAR. The use of efficient codons increases elongation rate. In some embodiments, the nucleic acid sequence is designed to exclude stretches of 20 nucleic acids of direct or inverted repeat sequences. Direct repeats are nucleotides sequences that consists of two or more repeats of a specific sequence, such that the repeats are present in multiple copies of a larger sequence. Generally, a direct repeat occurs when a sequence is repeated with the same pattern downstream. An inverted repeat is a single stranded sequence of nucleotides followed downstream by its reverse complement.

In some embodiments, the sequence of the nucleic acid encoding the CAR sequence is designed to exclude putative alternate splice sites. See, Wang et al., Gene 366(2):219-27 (2006), Lee et al., Annu. Rev. Biochem. 84:291-323 (2015), Baharlou et al., Sci. Rep. 8:5063-11 (2018), Jaganathan et al., Cell 176:535-548 (2019). In some embodiments, 3′ ends are modified to ensure the presence of proper transcription termination consensus sequences.

In some embodiments, the fully human VH domain amino acid sequence is reverse translated to generate an unoptimized DNA sequence, and then manually codon optimized. A nucleic acid insert may be produced by appending a short overlapping nucleic acid sequence of the expression vector 3′ insertion site to the 5′ end of the optimized nucleic acid encoding the fully human VH domain and a short overlapping nucleic acid sequence of the expression vector 5′ to the insertion site to the 3′ end of the optimized nucleic acid encoding the fully human VH domain. Additionally, a restriction enzyme recognition site (e.g., a NotI site), and an initiation site (e.g., a consensus Kozak Sequence) may be appended to the 5′ end of the optimized nucleic acid encoding the fully human VH domain and a spacer (e.g., 15 nucleic acid base pairs (bp)), and a restriction enzyme recognition site (e.g., a RsrII site) may be appended to the 3′ end of the optimized nucleic acid encoding the fully human VH domain.

The nucleic acid insert may then be analyzed for the presence of restriction enzyme recognition sites for the chosen restriction enzymes (e.g., NotI and RsrII) using commercially available software (Snapgene, G S L Biotech LLC, San Diego, CA). Internal restriction enzyme recognition sites identified may be removed by generating silent mutations according to codon usage frequency. Once the nucleic acid insert is free of internal restriction enzyme recognition sites, cryptic or alternative splice sites may be identified using a bioinformatic tool, e.g., the Alternative Splice Site Predictor ((ASSP) see, Wang et al., Gene 366(2):219-227(2006)). High scoring constitutive splice sites may be removed by generating silent mutations according to codon usage frequency and rechecked for the presence of both internal restriction enzyme recognition sites (e.g., NotI and RsrII) as well as new alternative splice sites. Nucleic acid inserts may then be checked for regions of undesired homology or long stretches (less than 20 nucleic acid bp) of direct or inverted repeat sequences via a DNA dot plot tool, e.g., YASS as well as IDT's E-block™ ‘Test Complexity’ tool (see, Noe et al., Nucleic Acids Research 33(2):W540-W543 (2005)). Regions of unwanted homology exceeding 20 nucleic acid bp may be removed by generating silent mutations according to codon usage frequency and rechecked for the presence of both internal restriction enzyme recognition sites as well as new alternative splice sites. Following removal of internal restriction enzyme recognition sites, regions of unwanted homology and high scoring constitutive splice sites, the entire open reading frame (ORF) may be translated and queried by BLAST to ensure all protein elements are coded as expected with a start codon and transcription termination consensus sequence (i.e., AATAAA).

### Expression Vectors

The CAR-encoding nucleic acids may be introduced into an immune cell by a suitable expression vector. An expression vector has elements that any expression vector may have in order to transport and effect expression of the CAR-encoding nucleic acid in an immune cell. Such elements include an origin of replication, a poly-A tail sequence, a selectable marker, and one or more suitable sites for the insertion of the nucleic acids, such as a multiple cloning site (MCS), one or more suitable promoters, each promoter operatively linked to the insertion sites of the nucleic acids and the selectable marker.

The term “promoter” as used herein refers to a non-coding nucleic acid that regulates, directly or indirectly, the transcription of a corresponding nucleic acid coding sequence to which it is operably linked, which in the context of the present disclosure is a CAR. A promoter may function alone to regulate transcription, or it may act in concert with one or more other regulatory sequences (e.g., enhancers or silencers, or regulatory elements that may be present in the gene construct or the expression vector). Promoters are located near the transcription start sites of genes, on the same strand and upstream on the DNA (towards the 5′ region of the sense strand). Promoters typically range from about 100-1000 base pairs in length.

The term “operatively linked” as used herein is to be understood that the CAR-encoding nucleic acid is spatially situated or disposed in the expression vector relative to a promoter to drive the expression of the CAR-encoding nucleic acid coding sequence.

In some embodiments, the expression vector contains a strong mammalian promoter, for example a cytomegalovirus (CMV) promoter, a simian virus 40 (SV40) early promoter, or promoters for β-actin or factor EF1α genes. In some embodiments, the promoter may have a core region located close to the transcription start site (TSS) and an enhancer, typically located farther upstream of the TSS. In some embodiments, the promoter is modified. One modification entails the removal of methylation sensitive sites (e.g., a cytosine nucleotide is followed by a guanine nucleotide, or “CpG”). Another modification entails the addition of a regulatory sequence that binds DNA methylation repressive transcriptional factors. In some embodiments, the expression vector includes A/T-rich, nuclear matrix interacting sequences, known as scaffold matrix attachment regions (S/MAR), which may enhance transformation efficiency and improve the stability of transgene expression.

In some embodiments, the expression vector is a viral vector, for example, a retroviral vector, a lentiviral vector, an adenoviral vector, a herpesvirus vector, an adenovirus, or an adeno-associated virus (AAV) vector. As used herein, the term “lentiviral vector” is intended to mean an infectious lentiviral particle. Lentivirinae (lentiviruses) is a subfamily of enveloped retrovirinae (retroviruses), that are distinguishable from other viruses by virion structure, host range, and pathological effects. An infectious lentiviral particle will be capable of invading a target host cell, including infecting, and transducing non-dividing cells and immune cells.

In some embodiments, the expression vector is a non-integrative and non-replicative recombinant lentivirus vector. The construction of lentiviral vectors has been described, for example, in U.S. Pat. Nos. 5,665,577, 5,981,276, 6,013,516, 7,090,837, 8,119,119 and 10,954,530. Lentivirus vectors include a defective lentiviral genome, i.e., in which at least one of the lentivirus genes gag, pol, and env, has been deleted or otherwise inactivated.

In other embodiments, the expression vector is a non-viral vector, representative examples of which include plasmids, mRNA, linear, single stranded (ss) DNA or linear double stranded (ds) DNA, minicircles, and transposon-based vectors, such as Sleeping Beauty (SB)-based vectors and piggyBac(PB)-based vectors. In yet other embodiments, the vector may include both viral and non-viral elements.

In some embodiments the expression vector is a plasmid. In addition to a promoter operatively linked to the CAR-encoding nucleic acid, the plasmid may also contain other elements e.g., that facilitate transport and expression of the CAR-encoding nucleic acid in an immune cell. The plasmid may be linearized with restriction enzymes, in vitro transcribed to produce mRNA, and then modified with a 5′ cap and 3′ poly-A tail.

In some embodiments, a carrier encapsulates the expression vector. The carrier may be lipid-based, e.g., lipid nanoparticles (LNPs), liposomes, lipid vesicles, or lipoplexes.

In some embodiments, the carrier is an LNP. In certain embodiments, an LNP includes two or more concentric bilayers separated by aqueous compartments. Lipid bilayers may be functionalized and/or crosslinked to one another. Lipid bilayers may include one or more ligands, proteins, or channels.

Lipid carriers, e.g., LNPs may include one or more cationic/ionizable lipids, one or more polymer conjugated lipids, one or more structural lipids, and/or one or more phospholipids. A “cationic” refers to positively charged lipid or a lipid capable of holding a positive charge. Cationic lipids include one or more amine group(s) which bear the positive charge, depending on pH. A “polymer conjugated lipid” refers to a lipid with a conjugated polymer portion. Polymer conjugated lipids include a pegylated lipids, which are lipids conjugated to polyethylene glycol. A “structure lipid” refers to a non-cationic lipid that does not have a net charge at physiological pH. Exemplary structural lipids include cholesterol, fecosterol, sitosterol, ergosterol, campesterol and the like. A “phospholipid” refers to lipids that have a triester of glycerol with two fatty acids and one phosphate ion. Phospholipids in LNPs assemble the lipids into one or more lipid bilayers. LNPs, their method of preparation, formulation, and delivery are disclosed in, e.g., U.S. Patent Application Publication Nos. 2004/0142025, 2007/0042031, and 2020/0237679, and U.S. Pat. Nos. 9,364,435, 9,518,272, 10,022,435, and 11,191,849.

Lipoplexes, liposomes, and lipid nanoparticles may include a combination of lipid molecules, e.g., a cationic lipid, a neutral lipid, an anionic lipid, polypeptide-lipid conjugates, and other stabilization components. Representative stabilization components include antioxidants, surfactants, and salts. Compositions and preparation methods of lipoplexes, liposomes, and lipid nanoparticles are known in the art. See, e.g., U.S. Pat. Nos. 8,058,069, 8,969,353, 9,682,139, 10,238,754, U.S. Patent Application Publications 2005/0064026 and 2018/0291086, and Lasic, Trends Biotechnol. 16(7):307-21 (1998), Lasic et al., FEBS Lett. 312(2-3):255-8 (1992), and Drummond et al., Pharmacol. Rev. 51(4):691-743 (1999).

### Cells

One aspect of the present disclosure is a genetically modified immune cell or a hematopoietic cell expressing one or more anti-BCMA CARs.

As used herein, the term “immune cell” refers to a cell of hematopoietic origin functionally involved in the initiation and/or execution of innate and/or adaptative immune response. As used herein, the term “hematopoietic cell” refers to a multipotent cell that can develop into all types of blood cells, including myeloid-lineage, lymphoid-lineage cells, and erythroid-lineage cells. Representative examples of immune cells include T cells, natural killer (NK) cells, macrophages, and dendritic cells. Representative examples of T cells include cytotoxic lymphocytes, T cells, cytotoxic T cells (CD8+ T cells), T helper cells (CD4+ T cells), αβ T cells and/or γδ T cells, Th17 T-cells, and NK T (NKT) cells. In some embodiments, the immune cells are CD8+ T cells. In some embodiments, the immune cells are CD4+ T cells. In some embodiments, the immune cells are central memory T cells. In some embodiments, the immune cells are stem cell-like central memory T cells. In some embodiments, the immune cells are NK cells. In some embodiments, the immune cells are macrophages. In some embodiments, the immune cells are dendritic cells.

Immune cells include cells derived from stem cells. The stem cells can be adult stem cells (e.g., induced pluripotent stem cells (iPSC)), embryonic stem cells, cord blood stem cells, progenitor cells, bone marrow stem cells, induced pluripotent stem cells, totipotent stem cells or hematopoietic stem cells. In some embodiments, the immune cells are derived from peripheral blood mononuclear cells (PBMC), cell lines, or cell bank cells. The collection, isolation, purification, and differentiation of cells from body fluids and tissues is known in the art. See, for example, Brown et al., PloS One 5:e11373-9 (2010), Rivera et al., Curr. Protoc. Stem Cell Biol. 54:e117-21 (2020), Seki et al., Cell Stem Cell 7:11-4 (2010), Takahashi et al., Cell, 126:663-76 (2006), Fusaki et al., Proc. Jpn. Acad. Ser. B Phys. Biol. Sci. 85:348-62 (2009), Park et al., Nature 451:141-6 (2008), and U.S. Pat. Nos. 10,214,722, 10,370,452, 10,428,309, 10,844,356, 11,141,471, 11,162,076, and 11,193,108 and U.S. Patent Application Publication Nos. 2012/0121544, 2018/0362927, 2019/0112577, and 2021/0015859.

Hematopoietic cells may be obtained from peripheral blood, bone marrow, and umbilical cord blood. Hematopoietic cells are distinguished by their ability of self-renewal (i.e., giving rise to more hematopoietic cells without differentiation), ability to undergo specific and large-scale differentiation into cells of various lineages. Hematopoietic cells may differentiate into one of several intermediate progenitor cells, including multipotent progenitors, oligopotent progenitors, and lineage restricted progenitor cells (Seita and Weissman, Wiley Interdiscip. Rev. Syst. Biol. Med. 2(6):640-653 (2010)). Mature effector cells that hematopoietic cells may differentiate into include platelets, erythrocytes, granulocytes, macrophages, dendritic cells, B cells, T cells, NK cells. Hematopoietic cells obtained from different sources typically have different differentiation potentials. For example, hematopoietic cells obtained from bone marrow may differentiate into any lineage blood cells, while hematopoietic cells obtained from the peripheral blood are in the myelosuppressive conditions of the blood. Therefore, hematopoietic cells in the peripheral blood may not readily form myeloid-lineage cells, but instead contribute to recovering damaged tissues (Lee and Hong, Int. J. Stem Cells 13(1):1-12 (2020)).

In some embodiments, the cells (immune cells or hematopoietic cells) are autologous with respect to the subject receiving the cells. In some embodiments, the cells are allogeneic to the subject receiving the cells, that is, the cells have a complete or at least partial HLA-match with the subject. For example, the cells or progenitors thereof can be obtained from one subject and administered to the same subject (autologous) or a different, compatible subject (allogeneic). In some embodiments, the cells contain one or more genetic modifications. In some embodiments, the cells are genetically modified by knocking out a component of the T cell receptor (TCR), including one or more of T cell receptor α constant (TRAC), T cell receptor βconstant (TRBC) 1, TRBC2, CD37, CD36, and CD3F. In some embodiments, the cells are genetically modified by knocking out one or more of β-2-microglobulin (B2MG), class II major histocompatibility complex transactivator (CIITA), HLA class I, and HLA class II. In some embodiments, the cells are genetically modified by knocking in (i.e., adding transgenes for) inhibitory molecules. Representative inhibitory molecules include major histocompatibility complex, class I, E (HLA-E) and CD47.

In some embodiments, the nucleic acids encode more than one CAR, e.g., a first and a second CAR, wherein each CAR contains an antigen recognition domain solely made up (i.e., consisting of) of a fully human, single VH domain that binds BCMA, but wherein they bind to different BCMA epitopes.

Methods of introducing the expression vectors containing the CAR-encoding nucleic acids into the cells are known in the art and detailed in Example 1. In some embodiments, a lentiviral expression vector is transduced into the cells.

In other embodiments, the method entails the use of gamma retroviral vectors. See, e.g., U.S. Pat. Nos. 9,669,049, 11,065,311, and 11,230,719. In some embodiments, the method entails the use of CRISPR to integrate (knock-in) the expression vector-delivered nucleic acids. In some embodiments, the method entails the use of Adeno-associated virus (AAV), dsRNA, ssDNA, or dsRNA to deliver the CRISPR- and CAR-encoding nucleic acids. See, e.g., U.S. Pat. No. 10,563,226, and U.S. Patent Application Publications 2019/0225991, 2020/0080108, and 2022/0186263.

In some embodiments, the method entails ex vivo or in vivo delivery of linear, circular, or self-amplifying mRNAs. See, e.g., U.S. Pat. Nos. 7,442,381, 7,332,322, 9,822,378, 9,254,265, 10,532,067, and 11,291,682. In some embodiments, the method entails the use of a transposase to integrate the expression vector-delivered nucleic acids into the cell's genome. See, e.g., U.S. Pat. Nos. 7,985,739, 10,174,309, 11,186,847, and 11,351,272. In some embodiments, the method entails the use of self-replicating episomal nano-vectors. See, e.g., U.S. Pat. Nos. 5,624,820, 5,674,703, 9,340,775.

In some embodiments, a plasmid with a CAR-encoding nucleic acid is transfected into the cells. CAR-encoding nucleic acid (e.g., plasmids, mRNA, linear ssDNA, or dsDNA) delivery into the cells may be performed by electroporation or by incorporation into LNP or exosomes. In some embodiments, the expression vector with the nucleic acids is delivered to a cell by lipofection. Lipofection is described, for example, in U.S. Pat. Nos. 5,049,386, 4,946,787; and 4,897,355.

### Pharmaceutical Compositions

Pharmaceutical compositions of the disclosure include therapeutically effective numbers of genetically modified cells (immune cells or hematopoietic cells) and a pharmaceutically acceptable carrier. Compositions may be provided as sterile liquid preparations, e.g., isotonic aqueous solutions, suspensions, emulsions, dispersions, or viscous compositions, which may be buffered to a selected pH. Liquid carriers include aqueous or non-aqueous carriers alike. Representative examples of liquid carriers include saline, phosphate buffered saline, a soluble protein, dimethyl sulfoxide (DMSO), polyol (e.g., glycerol, propylene glycol, liquid polyethylene glycol, and the like) and suitable mixtures thereof. In some embodiments, the liquid carrier includes a protein dissolved or dispersed therein, representative examples include serum albumin (e.g., human serum albumin, recombinant human albumin), gelatin, and casein. The compositions are typically isotonic, i.e., they have the same osmotic pressure as blood. Sodium chloride and isotonic electrolyte solutions (e.g., Plasma-Lyte®) may be used to achieve the desired isotonicity. Depending on the carrier and the cells, other excipients may be added, e.g., wetting, dispersing, or emulsifying agents, gelling and viscosity enhancing agents, preservatives and the like as known in the art.

The pharmaceutical composition may include more than one type of cell. Therefore, combinations of at least two different genetically modified cells may be used, wherein each type of cell is modified with the same or a different CAR-encoding nucleic acid. In some embodiments, the cells are a combination of immune cells and hematopoietic cells. In some embodiments, the cells are a combination of CD8⁺ T cells and CD4⁺ T cells. In some embodiments, the cells are a combination of T cells and NK cells.

### Methods of Use

In some aspects, the present disclosure is directed to treating cancer or an autoimmune disease that involves aberrant BCMA activity in a subject. The method entails administering to a subject in need thereof a therapeutically effective number of the genetically modified cells having a nucleic acid encoding one of the CARs described herein. The term “aberrant” when used in the context of BCMA gene product (RNA or protein) activity, refers to a decrease of BCMA gene expression, increase of BCMA gene expression (“overexpressed”), BCMA mutation, altered cellular location, or altered tissue location, as compared to a non-cancerous or non-diseased, normal physiological state.

The term “subject” (or “patient”) as used herein includes all members of the animal kingdom prone (or disposed) to or suffering from the indicated cancer or autoimmune disease. In some embodiments, the subject is a human. Therefore, a subject “having a cancer,” “having an autoimmune disease,” or “in need of” treatment according to the present disclosure broadly embraces subjects who have been positively diagnosed, including subjects having active disease who may have been previously treated with one or more rounds of therapy, and subjects who are not currently being treated (e.g., in remission) but who might still be at risk of relapse, and subjects who have not been positively diagnosed but who are predisposed to cancer or autoimmune disease (e.g., on account of the basis of prior medical history and/or family medical history, or who otherwise present with a one or more risk factors such that a medical professional might reasonably suspect that the subject was predisposed to cancer or autoimmune disease).

The terms “therapeutically effective number of immune cells” and “therapeutically effective number of hematopoietic cells” (each of which indirectly includes a corresponding amount of the CAR) as used herein refers to a sufficient number of the genetically modified cells that contain the CAR-encoding nucleic acid to provide the desired effect.

The effective number of the genetically modified cells for a given patient varies depending one or more factors that may include the age, body weight, type, location, and severity of the cancer or autoimmune disease and general health of the subject. Ultimately, the attending physician will decide the appropriate dose and dosage regimen. Typically, the cells will be given in a single dose. In some embodiments, the effective number of the genetically modified cells is about 1×10⁵ to about 1×10¹⁰ cells per subject. In some embodiments, the effective number of the genetically modified cells is about 1×10⁵ to about 6×10⁸ cells per kg of subject body weight.

The terms “treat”, “treating”, and “treatment” as used herein refer to any type of intervention, process performed on, or the administration of the genetically modified cells to the subject in need thereof with the therapeutic objective (“therapeutic effect”) of reversing, alleviating, ameliorating, inhibiting, diminishing, slowing down, arresting, stabilizing, or preventing the onset, progression, development, severity or recurrence of a symptom, complication or condition, or biochemical indicia associated with a cancer or autoimmune disease involving aberrant BCMA activity.

In some embodiments, the method entails treating a subject with an autoimmune disease involving aberrant BCMA activity. Representative autoimmune diseases that may be treatable in accordance with the present disclosure include lupus, myasthenia graves, immune thrombocytopenia (ITP), relapsed or refractory ITP, scleroderma, immune nephritis' Sjogren's syndrome, systemic lupus erythematosus (SLE), relapsed or refractory SLE, POEMS syndrome, pemphigus vulgaris, amyloidosis, autoimmune hemolytic anemia, and vasculitis.

In some embodiments, the method entails treating a subject with a cancer involving aberrant BCMA activity. Representative cancers that may be treatable in accordance with the present disclosure include hematological cancers and carcinomas.

Representative hematological cancers that may be treatable in accordance with the present disclosure include a plasma cell neoplasm (e.g., myeloma, multiple myeloma, relapsed or refractory multiple myeloma, high-risk multiple myeloma, plasma cell myeloma, extramedullary multiple myeloma, monoclonal gammopathy of unknown significance (MUGS), asymptomatic smoldering multiple myeloma, or solitary plasmacytoma), a lymphoma (e.g., Hodgkin's lymphoma, non-Hodgkin's lymphoma, Burkitt's lymphoma, plasmablastic lymphoma, plasmacytoid lymphoma, or diffuse large B-cell lymphoma), leukemia (e.g., relapsed or refractory acute B lymphocytic leukemia, or relapsed or refractory acute lymphoblastic leukemia).

Representative carcinomas that may be treatable in accordance with the present disclosure include Waldenstrom macroglobulinemia and glioblastoma (astrocytoma). In these embodiments, the therapeutic effect might include on or more art-recognized indicia of therapeutic efficacy, representative examples of which include prevention or prolongation of metastases, improvement in survival time, total/complete or partial remission of a cancer, e.g., no detectable cancer cells and less tumor cells or smaller tumors, respectively, a reduction in tumor cell number, or in a state of minimal residual disease (MRD). MRD is a state at which a cancer patient has a small number of cancer cells that remain in the body after treatment. In some embodiments, the cancer is relapsed or refractory. A relapsed cancer is a cancer that was treated, the treatment was stopped, and then the cancer returns after a disease-free period. A refractory cancer is a cancer that had previously been treated, responded to the that treatment, and subsequently stopped responding to that treatment. Relapsed and refractory cancers may have been in remission, under control, or in a state of MRD.

### Administration

Compositions containing a therapeutically effective number of the genetically modified cells (immune cells or hematopoietic cells) may be administered to a subject for the treatment of a cancer or autoimmune disease with aberrant BCMA activity by any medically acceptable route. The genetically modified cells are typically delivered intravenously, although they may also be introduced into other convenient sites (e.g., to an affected organ or tissue) or modes, as determined by an attending physician. Expansion and differentiation agents can be provided prior to, during or after administration of the cells to increase differentiation, expansion, or persistence of the genetically modified cells (e.g., T cells and NK cells).

### Combination Therapy

In some embodiments, the present methods may include co-administration of another anti-cancer agent.

The term “co-administered” includes substantially contemporaneous administration, by the same or separate dosage forms, or sequentially, e.g., as part of the same treatment regimen or by way of successive treatment regimens. Thus, if given sequentially, at the onset of administration of the second therapy, the first of the two therapies is, in some cases, still detectable at effective concentrations at the site of treatment. The sequence and time interval may be determined such that they can act together (e.g., synergistically to provide an increased benefit than if they were administered otherwise). For example, the therapeutics may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

Anti-cancer agents that may be used in combination with the inventive cells are known in the art. See, e.g., U.S. Pat. No. 9,101,622 (Section 5.2 thereof). An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of cancerous cells.

This process may involve contacting the cancer cells with recipient cells and the agent(s) or multiple factor(s) at the same time. This may be achieved by contacting the cancer cells with a single composition or pharmacological formulation that includes both agents, or by contacting the cancer cells with two distinct compositions or formulations, at the same time, wherein one composition includes recipient cells and the other includes the second agent(s).

In some embodiments, the genetically modified cells of the present disclosure are used in conjunction with chemotherapeutic, radiotherapeutic, immunotherapeutic intervention, targeted therapy, pro-apoptotic therapy, or cell cycle regulation therapy. In some embodiments, the genetically modified cells of the present disclosure are administered after the subject receives lymphodepletion chemotherapy. In some embodiments, the lymphodepletion chemotherapy is melphalan. In some embodiments, the lymphodepletion chemotherapy includes one or both of fludarabine (Flu) and cyclophosphamide (Cy). In some embodiments, the subject receives a stem cell transplant after the lymphodepletion chemotherapy.

In some embodiments, the genetically modified cells of the present disclosure are used in conjunction with an additional active agent that targets myeloma cells. In some embodiments, the additional active agent binds CD3, CD16A, CD16B, CD38, CD44, CD138, CD229, SLAM Family Member 7 (SLAMF7), integrin 37 (ITGB7), Natural Killer Group 2D (NKG2D), NK Cell Activating Receptor (NKp44), also known as Natural Cytotoxicity Triggering Receptor 2 (NCR2) and CD336, NKp46, also known as NCR1 and CD335, or cereblon E3 ligase. In some embodiments, the additional active agent is an anti-BCMA and anti-CD16 bispecific antibody, for example, AFM26 produced by Affimed Therapeutics or RO7297089 produced by Genentech. In some embodiments, the additional active agent is a trispecific antibody, for example, the trispecific anti-BCMA, anti-CD200, and anti-CD16 antibody aTriFlex produced by Affimed Therapeutics. In some embodiments, the additional active agent is the trifunctional natural killer (NK) cell engager SAR443579 produced by Sanofi and Innate Pharma which targets CD123 and co-engaging NKp46 and CD16A on NK cells.

In some embodiments, the additional active agent is one or more of bortezomib, carfilzomib, ixazomib, lenalidomide, pomalidomide, thalidomide, dexamethasone, prednisone, elotuzumab, daratumumab, isatuximab, and mezigdomide, iberdomide, talquetamab, monalizumab, AMG420, and AMG701.

In some embodiments, the genetically modified cells of the present disclosure are used in conjunction with a therapeutically effective amount of an additional active agent that targets lymphoma cells. In some embodiments, the additional active agent is one or more of rituximab, mosunetuzumab, and blinatumomab.

In some embodiments, the genetically modified cells of the present disclosure are used in conjunction with a therapeutically effective amount of a gamma secretase inhibitor. In some embodiments, the gamma secretase inhibitor is one or more of Avagacestat, Begacestat, Crenigacestat, Iminostilbene, Itanapraced, Nirogacestat, L-685458, Semagacestat, and Tarenflurbil.

### Immunotherapy

Immunotherapy, including immune checkpoint inhibitors may be employed to treat a diagnosed cancer. Immune checkpoint molecules include, for example, PD1, CTLA4, KIR, TIGIT, TIM-3, LAG-3, BTLA, VISTA, CD47, and NKG2A. Clinically available examples of immune checkpoint inhibitors include durvalumab (Imfinzi®), atezolizumab (Tecentriq®), and avelumab (Bavencio®). Clinically available examples of PD1 inhibitors include nivolumab (Opdivo®), pembrolizumab (Keytruda®), and cemiplimab (Libtayo®).

### Chemotherapy

Anti-cancer therapies also include a variety of combination therapies with both chemical and radiation-based treatments. Combination chemotherapies include, for example, Abraxane®, altretamine, docetaxel, Herceptin®, methotrexate, Novantrone®, Zoladex®, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, Taxol®, gemcitabien, Navelbine®, farnesyl-protein tansferase inhibitors, transplatinum, 5-fluorouracil, vincristine, vinblastine and methotrexate, or any analog or derivative variant of the foregoing and also combinations thereof.

### Radiotherapy

Anti-cancer therapies also include radiation-based, DNA-damaging treatments. Combination radiotherapies include what are commonly known as gamma-rays, X-rays, and/or the directed delivery of radioisotopes to tumor cells which cause a broad range of damage on DNA, on the replication and repair of DNA, and on the assembly and maintenance of chromosomes. Dosage ranges for radioisotopes vary widely, and depend on the half-life of the isotope, the strength and type of radiation emitted, and the uptake by the neoplastic cells and will be determined by the attending physician.

Radiotherapy may include external or internal radiation therapy. External radiation therapy involves a radiation source outside the subject's body and sending the radiation toward the area of the cancer within the body. Internal radiation therapy uses a radioactive substance sealed in needles, seeds, wires, or catheters that are placed directly into or near the cancer.

These and other aspects of the present disclosure will be further appreciated upon consideration of the following working examples, which are intended to illustrate certain embodiments of the disclosure but are not intended to limit its scope, as defined by the claims.

### EXAMPLES

### Example 1: Materials and Methods

Cell lines and Donor T cells. The human MM cell line OPM2 was obtained from Deutsche Sammlung von Mikroorganismen und Zellkulturen (DMSZ) and maintained in RPMI and 10% fetal bovine serum (FBS) (Gibco, Life Technologies, Gaithersburg, MD). NIH 3T3 Fibroblast cells were maintained in DMEM and 10% FBS (Gibco, Life Technologies). To generate GFP/luciferase-positive target cells or to express BCMA in NIH-3T3s, cells were stably transduced with gamma retrovirus expressing the cDNA. These target cells were then sorted into single-cell clones in 96-well plates, and expanded to generate clonal populations, which were confirmed by flow cytometry for use in all experiments. Human T cells were obtained from the peripheral blood of healthy donors (MGB Crimson Core Blood Bank; study #T0761).

T cells were stimulated with CD3/CD28 TransAct (Miltenyi, #130-111-160) at a 1:100 ratio for 48 hours and grown in the presence of IL-2±IL-7 and IL-15 (NIH BRB Preclinical Biologics Repository).

Lentiviral plasmid construction and viral production and transduction. The amino acid sequences of the BCMA binders, after codon optimization and checking for predicted alternative splice sites, were cloned into a modular bicistronic lentiviral CAR backbone that includes the 4-1BB/CD3ζ and dsRedE2 fluorescent reporter, separated by a T2A sequence. All the generated plasmids were sequence verified by whole plasmid sequencing (plasmidsaurus) or Sanger sequencing (Genewiz).

To design and generate an optimal anti-BCMA CAR DNA construct from a discovered anti-BCMA single domain binder, the amino acid sequence of the fully human, anti-BCMA VH binder was first reverse translated to generate an unoptimized DNA sequence. Following reverse translation, this unoptimized DNA sequence of the fully human, anti-BCMA VH binder was manually codon optimized. A short overlapping sequence to the CAR recipient expression vector containing a NotI restriction enzyme recognition site, a consensus Kozak Sequence and Igκ signal peptide were appended to the 5′ end of the newly optimized fully human, anti-BCMA VH binder sequence and a short overlapping region to CAR recipient expression vector encoding 15 bp of a spacer and a RsrII restriction enzyme recognition site was appended to the 3′ end to generate the CAR insert. The CAR insert was then analyzed for the presence of recognition sites for NotI and RsrII—the chosen restriction enzymes used for subcloning into the CAR recipient expression vector—using commercially available software (Snapgene, GSL Biotech LLC, San Diego, CA). Any internal restriction enzyme recognition sites were removed by generating silent mutations according to codon usage frequency.

Once the CAR insert was free of internal restriction enzyme recognition sites, cryptic or alternative splice sites were identified using the Alternative Splice Site Predictor (ASSP) bioinformatic tool (See, Wang et al., Gene 366(2):219-227(2006)). Any high scoring constitutive splice sites were removed by generating silent mutations according to codon usage frequency and rechecked for the presence of both internal NotI and RsrII recognition sites as well as new alternative splice sites. Inserts were then checked for regions of undesired homology or long stretches (greater than 20 bp) of direct or inverted repeat sequences via a DNA dot plot tool, YASS, as well as IDT's E-block™ ‘Test Complexity’ tool (see, Noe et al., Nucleic Acids Research 33(2):W540-W543 (2005)). Regions of unwanted homology exceeding 20 bp were removed by generating silent mutations according to codon usage frequency and rechecked for the presence of both internal restriction enzyme recognition sites as well as new alternative splice sites. Following removal of internal restriction enzyme recognition sites, regions of unwanted homology and high scoring constitutive splice sites, the entire ORF was translated and queried by BLAST to ensure all protein elements were coded as expected with a start codon and transcription termination consensus sequence (i.e., AATAAA). Optimized inserts that were free of predicted sequence liabilities were synthesized as eBlock™ gene fragments by IDT (Coralville, IA) and subcloned into the CAR recipient expression vector to generate the CAR plasmid.

The lentivirus was packaged and produced based on a protocol adapted from a previously described publication (Salmon and Trono, Curr. Protoc. Hum. Genet. 54(1) Chapter 12:12-10). In brief, 293-based packaging cells were seeded onto a 150 mm tissue-culture treated plates (8×10⁶ cells/plate) for 24 h, then transfected with plasmids encoding the CAR, pMD.2G encoding VSV-G envelope and a packaging expression vector psPAX2, using the transfection reagent, Polyethylenimine (PEI, Polysciences, #23966) following the manufacturer's instructions. The viral supernatant was harvested at 24 h and 48 h, filtered and concentrated by ultracentrifugation at 25000 rpm for 2 h at 4° C. Supernatant was discarded, and the pellet was then resuspended in 100 μL of serum free media, shaken overnight at 4° C. and aliquots were then stored at −80° C.

T cells were spinoculated at 2000×g for 1 h with lentivirus (1% by volume) along with Lentiboost-B (Sirion Biotech), 2 days after their activation. Transduction efficiency was determined by flow cytometric analysis on the Northern Lights (Cytek Bio) of the fluorescent reporter, DsRedE2, on day 4 after transduction. All experiments were normalized for CAR⁺ viable cells.

Cytotoxicity. 3T3 overexpressing BCMA stably transduced with ffLuc via retrovirus as described above were used as target cells. 10,000 target cells were plated in 96-well plates in triplicate with CAR⁺ T cells at the indicated effector-to-target (E:T) ratios and incubated for 24 h. An ATP-dependent assay was performed where % cytotoxicity=(BLI_{Mock}−BLI_{Sample})/BLI_{Mock}, BLI_{Mock}=mean target cell alone value of that experiment. Bioluminescence was read on a BioTek Cytation 5.

Tonic signaling assay. A Jurkat T cell leukemia Nur77-GFP reporter cell line was generated by inserting a 2A-GFP sequence in-frame with the endogenous Nur77 gene by homologous recombination. This Jurkat Nur77-GFP line was further engineered to express various anti-BCMA CAR-2A-RFP bicistronic constructs. Cells were plated either alone or 2:1 with 3T3 BCMA cells for 20 h. Antigen-independent (tonic signaling) and antigen-dependent activation were assessed by measuring changes in GFP expression by flow cytometry. Signaling of transduced cells was calculated as the ratio of GFP⁺RFP⁺ cells to total RFP⁺ cells.

In vivo studies. Studies were performed in accordance with Dana Farber Cancer Institutional Animal Care and Use Committee-approved protocol (20-010). Six-week-old NSG (NOD.Cg-Prkd^{scid} Il2rg^{tm1Wjl}/SzJ) mice (Jackson Labs; Bar Harbor, ME) were injected with the bone marrow tropic cell line OPM2-ffLuc, at a dose of 1×10⁶ cells via tail vein. Tumor engraftment was confirmed by baseline bioluminescent imaging before cellular therapy. Two weeks after the tumor engraftment, a single dose of 0.5×10⁶ human anti-BCMA CAR T cells was injected via tail vein. In vivo imaging was preformed after injection of D-luciferin (Millipore-Sigma; Darmstadt, Germany) and analyzed with Living Image software (PerkinElmer; Waltham, MA). Studies were planned with the minimum number of animals per treatment group to reproducibly observe statistically significant differences (n=5 per experiment). Statistical significance between Kaplan-Meier curves was determined with log-rank (Mantel-Cox) test. Statistical significance of BLI quantification was determined using Student's t test.

### Example 2: Generation of Fully Human HCAb Antibodies Against BCMA

Harbour HCAb mice were utilized to produce HCAb antibodies. Harbour HCAb mice produce humanized, dimeric antibodies made up of two heavy chains, each with a VH, CH2, and CH3 regions.

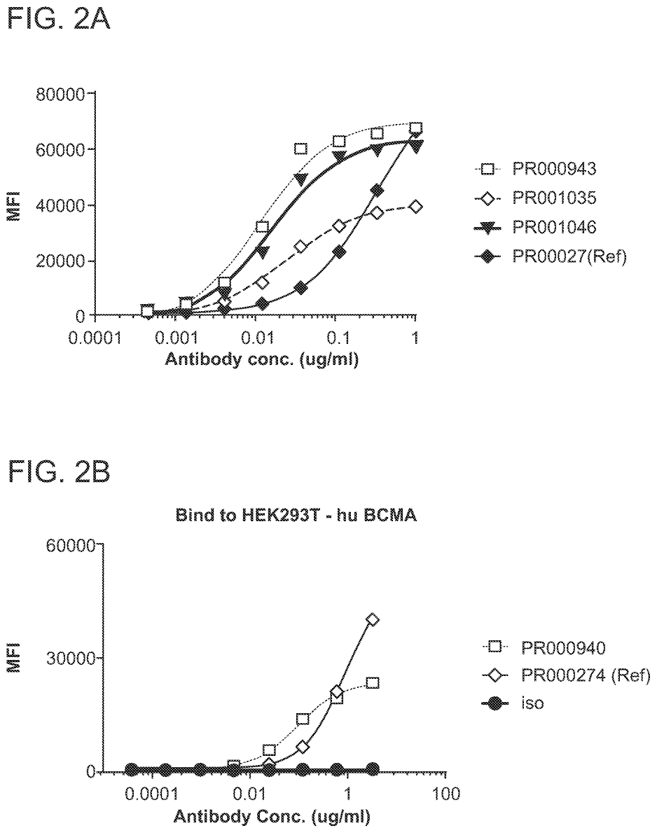

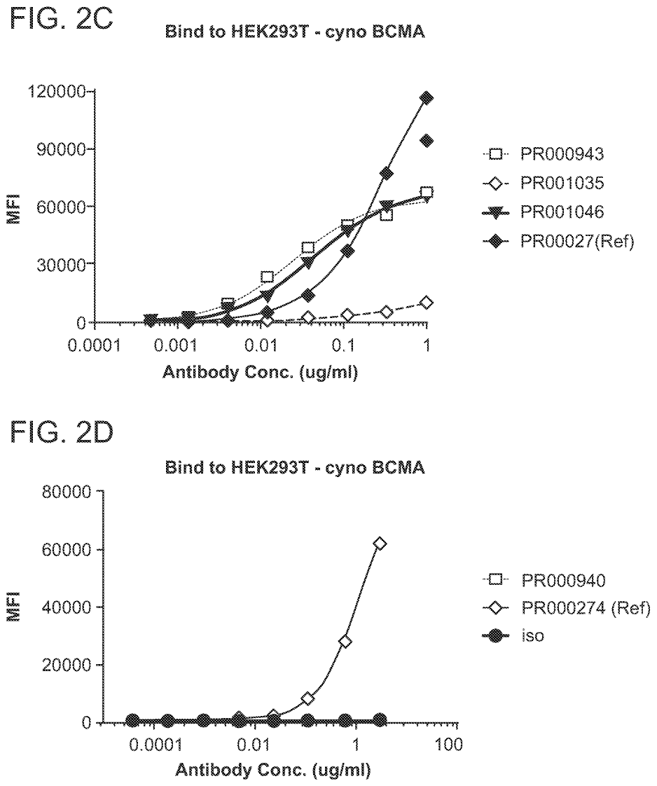

HCAb antibodies were isolated from mice and characterized. The characterized antibodies include PR000940, PR000943, PR001035, PR001046, as well as the reference antibody PR000274. The humanized HCAbs are described in U.S. Patent Application Publication 2023/0322953. The reference, positive control antibody PR0000274 is clone CA8-J6M0 and is described in U.S. Pat. No. 9,273,141. Clone CA8-J6M0 is a clinically validated anti-BCMA antibody. The antibodies sequences were determined and are listed in Table 1-Table 4. The newly raised HCAbs antibodies bind to human BCMA cell lines, with comparable binding strength as the reference antibody PR000274 (FIG. 2A-FIG. 2B). Two antibodies, PR000943 and PR001046, had good cross-reactivity (FIG. 2C-FIG. 2D), exemplified by binding to HEK 293T cells that express the cynomolgus (cyno) monkey BCMA protein.

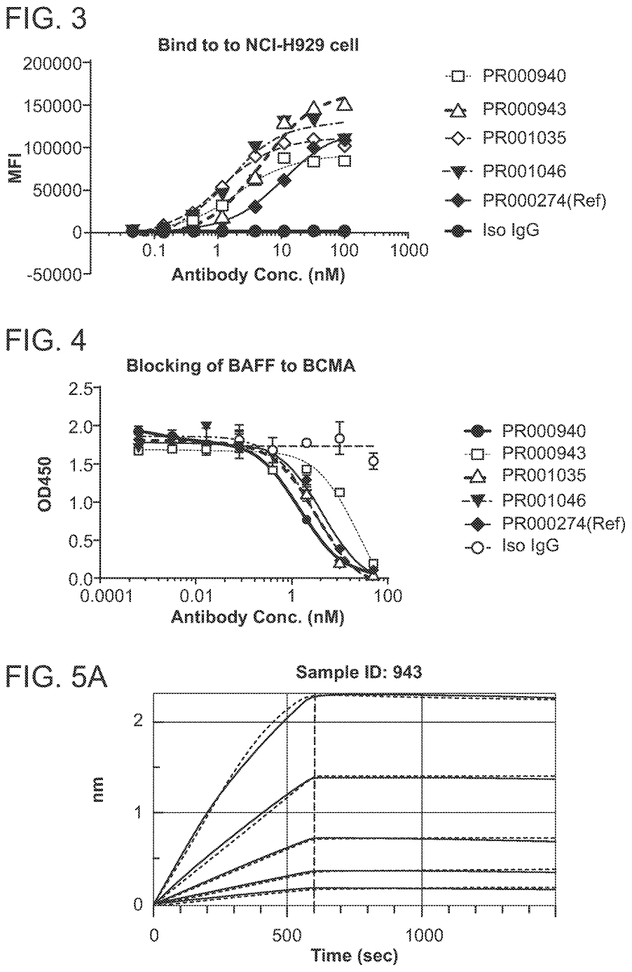

Furthermore, the fully human HCAb antibodies PR000940, PR001035, and PR001046 bound strongly to NCI-H9292 cells (FIG. 3) as compared to PR000274 reference. As shown in FIG. 4 and quantified in Table 10, the HCAb antibodies PR000940, PR000943, PR001035, and PR001046 also blocked binding of BAFF to BCMA cells.

TABLE 10
HCAb antibodies have BAFF blocking function

| | PR000940 | PR000943 | PR001035 | PR001046 | PR000274 |
|---|---|---|---|---|---|
| IC50 | 1.407 | 24.34 | 3.047 | 2.940 | 4.356 |
| Span | 1.796 | 2.188 | 1.917 | 1.985 | 1.862 |

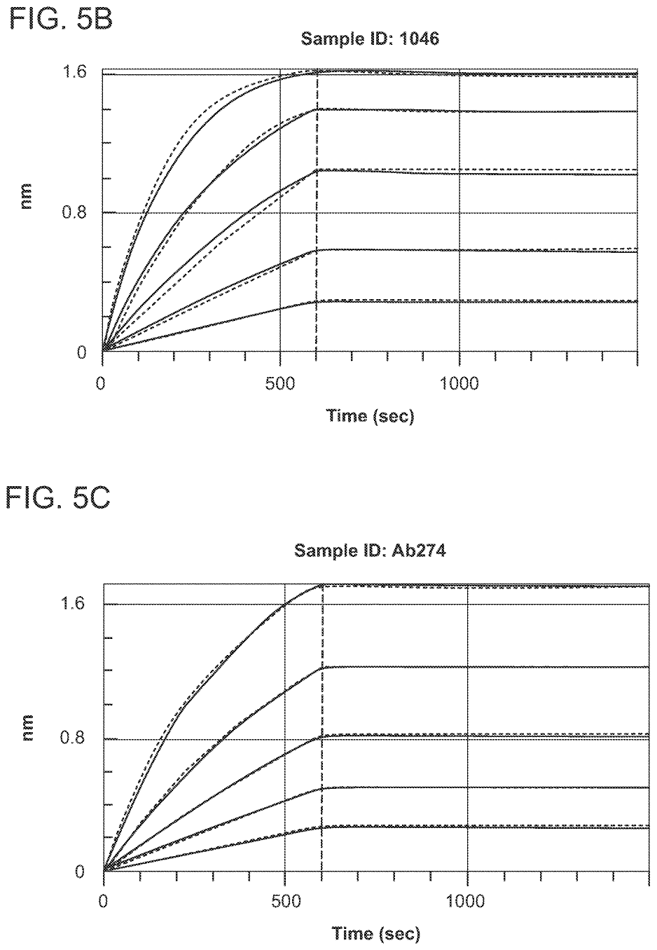

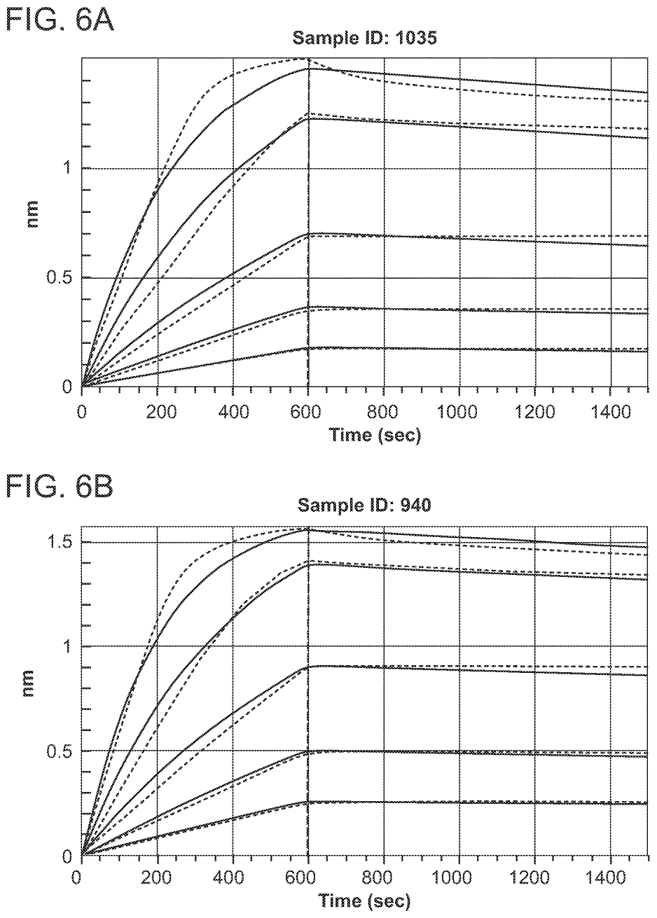

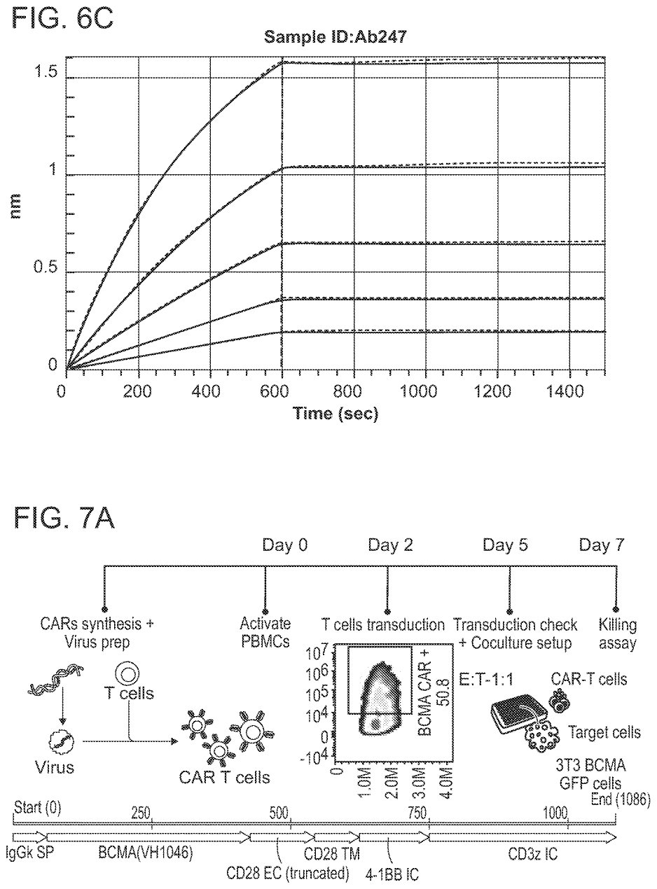

The HCAb antibodies have strong binding affinity for BMCA target epitopes. The binding affinity of PR000943 and PR001046 as compared to the reference PR000274 are illustrated in FIG. 5A-FIG. 5C and quantified in Table 11. The binding affinity of HCAb antibodies PR000940, PR001035 and the reference PR000274 are illustrated in FIG. 6A-FIG. 6C and quantified in Table 12. The sensor was streptavidin (SA), loaded with hBCMA-his-biotin from Acro catalog number BCA-H82E4, and 50 nM antibody with a 1:2 dilution factor. Association times in FIG. 5A-FIG. 5C were each 600 s and dissociation times were each 900 s. Fit curves were concentrations of 12.5 nM, 6.25 nM, 3.13 nM, 1.56 nM and 0.78 nM.

TABLE 11
HCAb antibodies have strong binding affinity

| Sample ID | KD (M) | KD Error | Kon* | Kon Error | Kdis† | Kdis Error | Full R\hat{ }2 |
|---|---|---|---|---|---|---|---|
| PR000943 | 1.62e−10 | 9.38e−12 | 1.19e+05 | 9.03e+02 | 1.93e−05 | 1.11e−06 | 0.9986 |
| PR001046 | 2.53e−11 | 2.21e−12 | 4.23e+05 | 1.26e+03 | 1.07e−05 | 9.37e−07 | 0.9983 |
| PR000274 | <1.0e−12 | <1.0e−12 | 2.28e+05 | 1.39e+02 | <1.0e−7 | | 1 |
| (Ref) | | | | | | | |

*1/Ms
†1/s

TABLE 12
HCAb antibodies have strong binding affinity

| Sample ID | KD (M) | KD Error | Kon* | Kon Error | Kdis† | Kdis Error | Full R\hat{ }2 |
|---|---|---|---|---|---|---|---|
| PR000940 | 1.52e−10 | 4.35e−12 | 4.01e+05 | 2.18e+03 | 6.09e−05 | 1.71e−06 | 0.995 |
| PR001035 | 2.76e−10 | 7.26e−12 | 3.25e+05 | 2.49e+03 | 8.98e−05 | 2.26e−06 | 0.9926 |
| PR000274 | <1.0e−12 | 2.38e−12 | 1.95e+05 | 4.33e+02 | <1.0e−7 | | 0.9997 |
| (Ref) | | | | | | | |

*1/Ms
†1/s

### Example 3: Cytotoxicity of VH-Only BCMA CARs

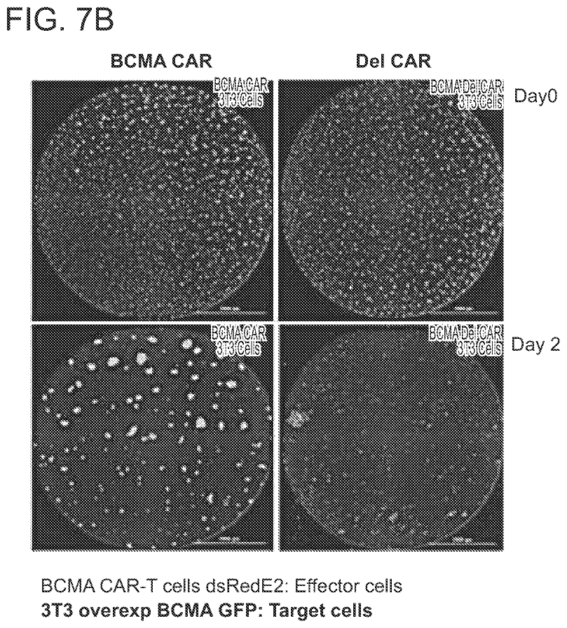

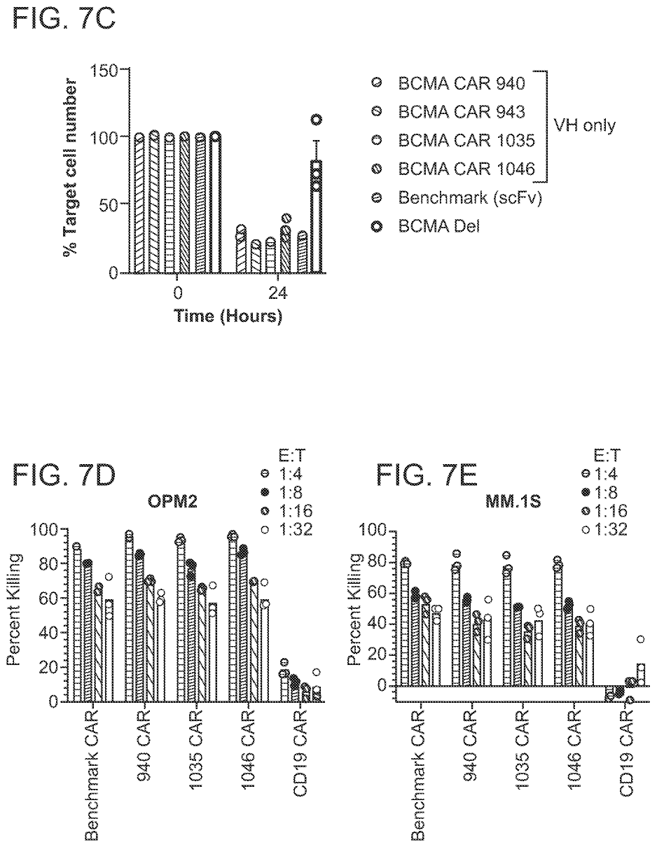

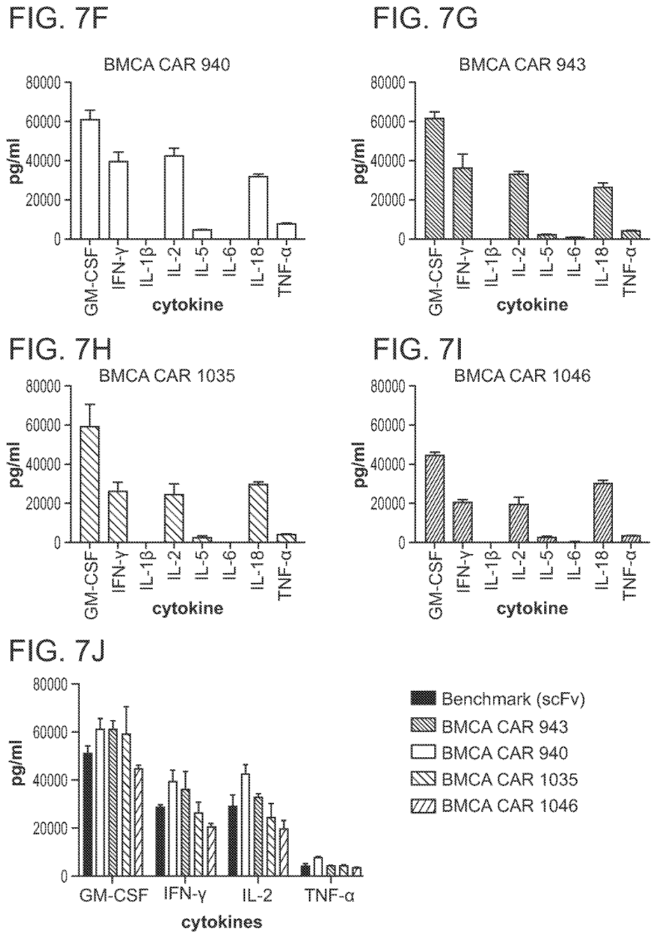

To confirm VH-only anti-BCMA CAR cell cytotoxicity, T cells were isolated from healthy donor PBMCs and transduced with CAR constructs and transduction confirmation, coculturing, and detection of CAR T cell killing were performed (FIG. 7A). CAR T cells containing antigen recognition domains derived from PR000940, PR000943, PR001035, and PR001046, abbreviated as BCMA CAR 940, BCMA CAR 943, BCMA CAR 1035, and BCMA CAR 1046, respectively, were produced. Transduction was confirmed by flow cytometry either for direct BCMA CAR expression or by a marker (e.g., dsRed2).

VH-only anti-BCMA CAR cells were incubated at a 1:1 effector:target (E:T) ratio for 24 h in 96 well plates with the mouse embryonic fibroblast 3T3 cell line target cells. Cytotoxicity was confirmed by clearance of GFP+ target cells. Representative images show that VH-only anti-BCMA CAR cells killed the 3T3 target cells, as seen by loss of GFP+ cells after 2 days of co-culture (FIG. 7B, left two images). Expansion of the VH-only anti-BCMA CAR cells were also seen in (red). Negative control, cells expressing a CAR with the signaling domain deleted (abbreviated Del CAR), did not kill the 3T3 cells (FIG. 7B, right two images). Del CAR cells did not expand.

Quantification of the percentage of target cell number remaining after the killing assay is shown in FIG. 7C. All four VH-only anti-BCMA CAR cell groups tested effectively killed the 3T3 target cells (first four bars, left to right). Negative control CAR deleted cells (abbreviated BCMA Del) did not kill the target cells. Positive control (i.e., benchmark) anti-BCMA cells also killed the target cells. The positive control CAR cells, which are described in Works et al., Mol. Cancer Ther. 18:2246-2257 (2019) and U.S. Patent Application Publication 2021/0324100, have an extracellular BCMA-binding single-chain variable fragment antigen recognition domain.

BCMA VH-only CARs induce effective cytotoxicity at low E:T ratios. Quantification of the percentage of target cell number remaining after the killing assay is shown in FIGS. 7D and 7E. VH-only anti-BCMA CAR cell groups tested effectively killed either OPM2 (left) or MM.1S (right) target myeloma cells. Irrelevantly targeted (CD19) negative control CAR did not kill the target cells. Positive control anti-BCMA CAR T cells, also described in Works, et al., supra and U.S. Patent Application Publication 2021/0324100, and which have an extracellular BCMA-binding single-chain variable fragment antigen recognition domain, also killed the target cells.

BCMA VH-only CARs produce cytokines when co-cultured with target cells. Cytokine profiles of four novel VH-only anti-BCMA CAR T cells are shown in FIGS. 7F-7I. VH-only anti-BCMA CAR T cells produced GM-CSF, IFN-7., IL-2, IL-18, and, to a lesser extent, IL-5, and TNF-α (FIGS. 7F-7I) after co-culture with endogenous BCMA-expressing OPM2 myeloma cells at a E:T ratio of 1:1. FIG. 7J shows the comparative cytokine secretion of four novel BCMA CAR T cells co-cultured with endogenously BCMA-expressing OPM2 myeloma cell line at a 1:1 E:T ratio. Control CAR T cells expressing a CAR containing an antigen recognition domain derived from the reference antibody PR00274, produced cytokines at comparable levels as the VH-only anti-BCMA CAR T cells.

### Example 4: Tonic Signaling

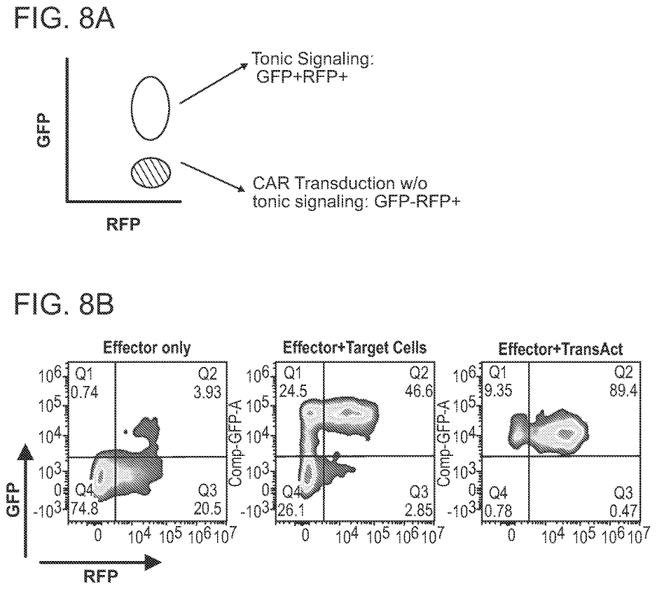

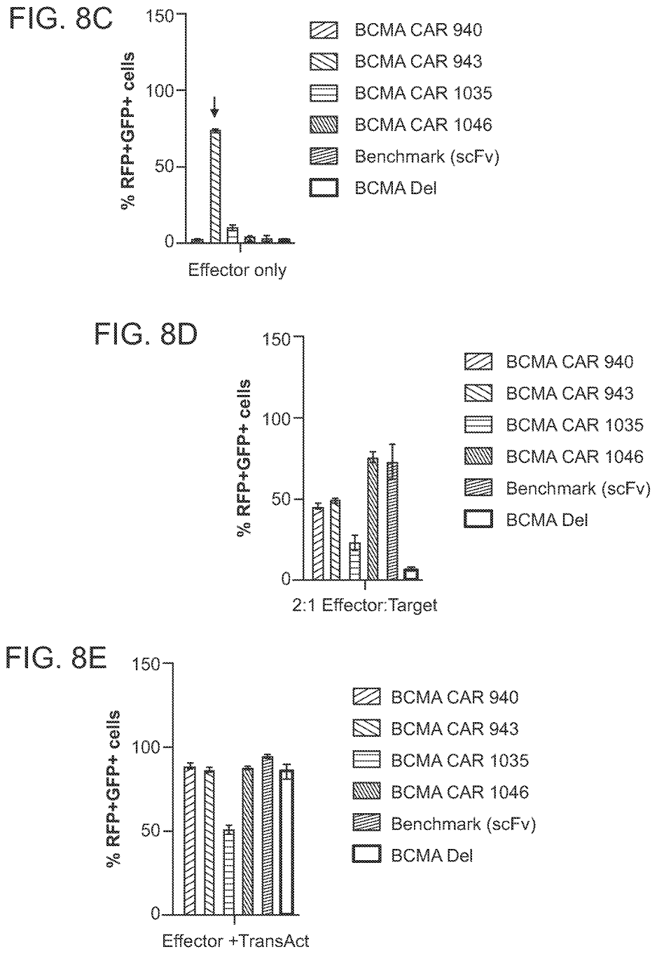

Several VH-only anti-BCMA CAR cells showed minimal tonic (antigen independent) signaling while maintaining the property to induce antigen specific signaling. A tonic signaling reporter cell line was generated that can identify CAR cells with high antigen independent signaling. Antigen independent (tonic) signaling has been demonstrated by most investigations to contribute to activation induced cell death (AICD) or pre-mature dysfunction, potentially limiting in vivo or clinical efficacy of a CAR candidate. A reporter cell line for CAR signaling was developed by knocking in GFP in-frame (using a 2A ribosomal skipping element) with the Nuclear Receptor Subfamily 4, Group A, Member 1 (NR4A1) gene, also known as Nuclear Hormone Receptor 77 (Nur77). NR4A1 is transcribed early, downstream from CD3ζ signaling. In this reporter line, RFP signal indicates successful CAR transduction into the cells and GFP signal indicates downstream CAR-signaling (FIG. 8A). GFP is co-transcribed with NF4A1, an early indicator of CD3ζ signaling. The RFP+GFP+ double positive population indicates CAR induced signaling, which reflects tonic signaling in the absence of target cells. Representative flow cytometry plot of example showing minimal tonic signaling in the Effector only group (FIG. 8B, left), with only 3.93% of cells being RFP+GFP+ double positive. Robust, antigen dependent signaling is seen when the VH-only anti-BCMA CAR effector cells are co-cultured with target cells, with 46.6% cells expressing both RFP+ and GFP+ (FIG. 8B, middle). CD3/CD28 beads represent a positive control for T cell activation, with 89.4% of cells expressing both RFP+ and GFP+ (FIG. 8B, right).

Tonic, antigen-independent signaling of four VH-only anti-BCMA CAR cells was investigated. Only one, PR000943 VH-only anti-BCMA CAR cells showed RFP+GFP+ tonic signaling cells when incubated with effector cells only (FIG. 8C). The VH-only anti-BCMA CAR cells were confirmed to function when incubated with target cells at a 2:1 effector:target ratio (FIG. 8D) and when incubated with CD3/CD28 beads (TransAct) (FIG. 8E). PR001046 VH-only anti-BCMA CAR cells showed the highest antigen specific signaling after co-culture with target cells (FIG. 8D).

### Example 5: VH-Only Anti-BCMA CAR Cells in a Bone Marrow Tropic MM Xenograft

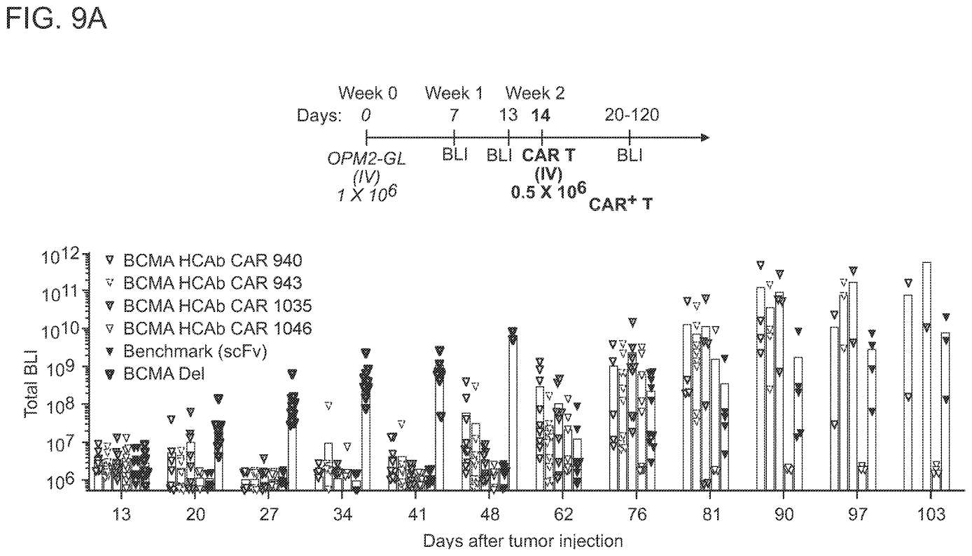

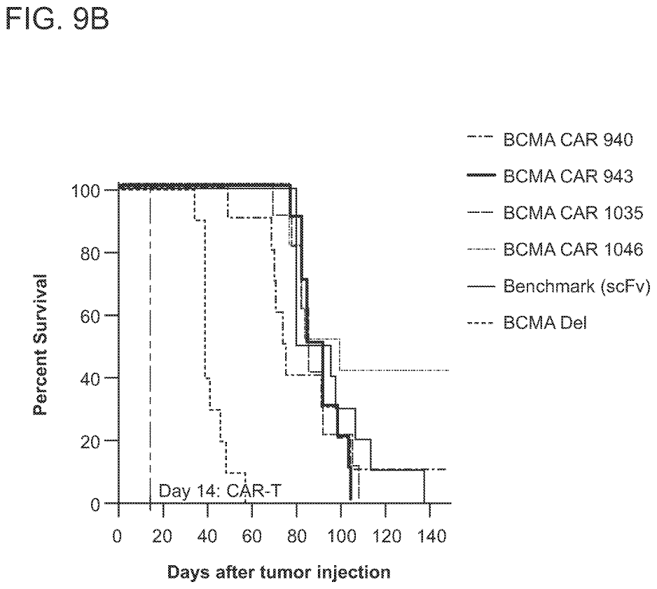

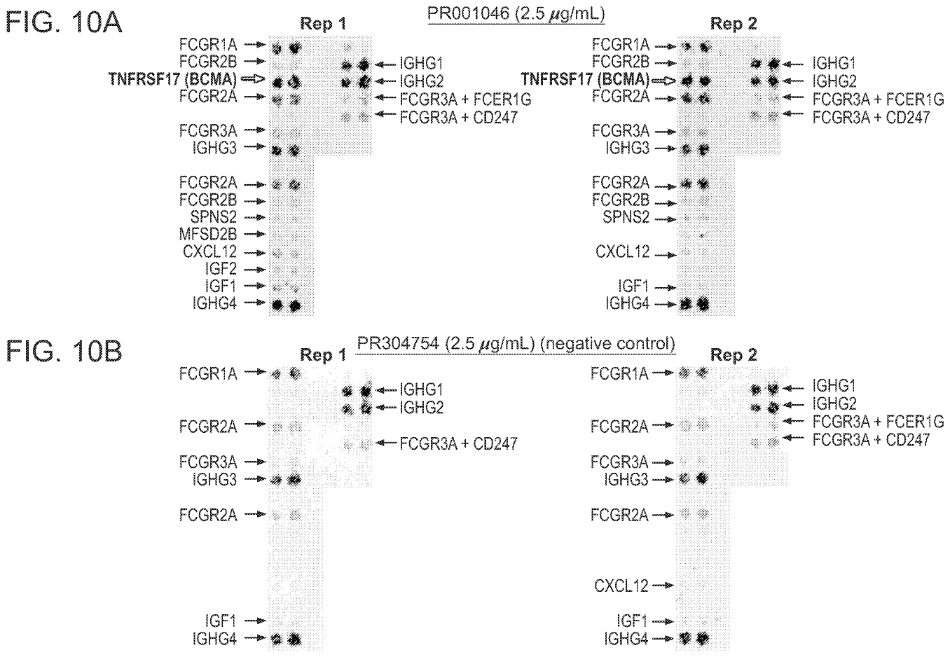

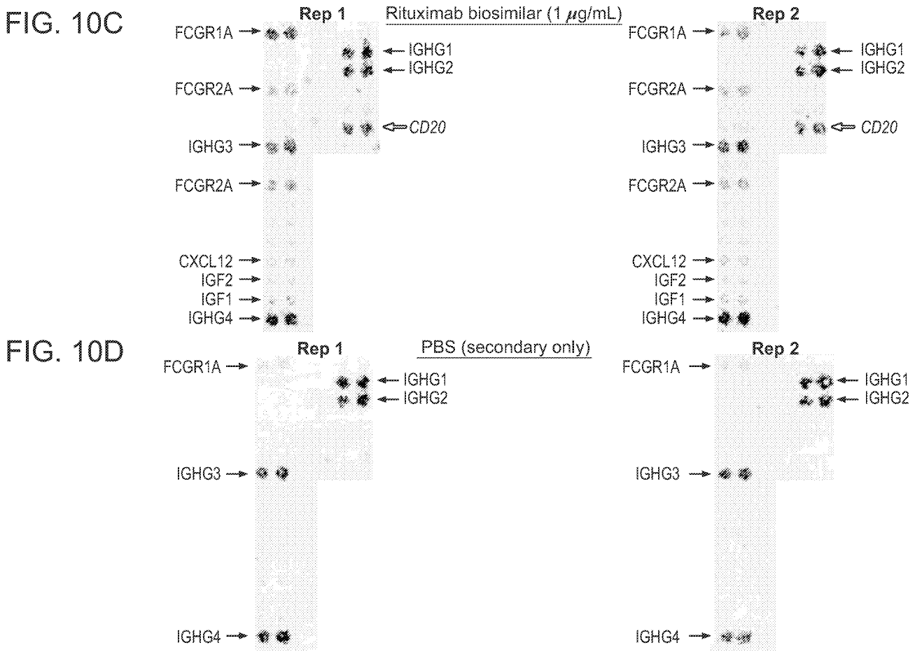

Four VH-only anti-BCMA CARs, each incorporating different VH-only clones (940, 943, 1035, 1046), were investigated for tumor control in a bone marrow tropic MM xenograft mouse model. Mice were injected as illustrated in FIG. 9A. All four VH-only anti-BCMA CARs were shown to be highly active compared to negative control CAR (41BB/CD3z signaling deleted (BCMA Del)), quantified as bioluminescent tumor imaging mean (FIG. 9A). FIG. 9B shows Kaplan-Meier curves of mouse survival. VH-only anti-BCMA clone 1046 CAR had the highest antigen-specific activation, and the most rapid (day 20) disease control (FIG. 9A). Similarly, mice treated with the VH-only anti-BCMA clone 1046 CAR T cells also had the largest percentage of durable remissions when compared to other VH-only CAR T cells of the positive refence control CAR T cells (FIG. 9B). Therefore, CAR T cells containing the VH-only anti-BCMA clone 1046 had the highest in vivo survival (FIG. 9B), while they also had the lowest cytokine release (FIG. 7F), attributes which may lead to a uniquely high therapeutic window.

### Example 6: On-Target VH-Only Anti-BCMA CAR Specificity

In order to screen the on-target specificity of one of the VH-only anti-BCMA-targeted CARs, the Retrogenix Cell Microarray Technology platform was employed, which is a high-throughput platform for screening binding interactions between a test molecule and about 6100 human surfaceome proteins. A CAR VH-only binder reformatted as a VH-only human IgG1, known as PR001046, was used. Preliminary analysis with untransfected HEK293 cells and BCMA-overexpressing HEK293 cells showed that 2.5 μg/mL of PR001046 VH-only human IgG1 test antibody was a suitable concentration and was used for subsequent full library screening. An AlexaFluor647 anti-hIgGFc detection antibody was used as a secondary antibody.

In the library screening, the CAR-derived PR001046 VH-only human IgG1 test antibody was screened for binding against fixed human HEK293 cells, individually expressing 6105 full-length human plasma membrane (PM) proteins, evidence of membrane (M), secreted proteins (S), and cell surface-tethered secreted (TS) proteins plus a further 400 human heterodimers (HD). The screen identified 21 library interactions, primarily a strong interaction to BCMA (also known as TNF receptor super family member 17 (TNFRSF17)) and weak interactions with members of the Fc gamma receptor (FcTR) family (Table 13). Each library interaction was re-expressed, along with 2 control receptors, and re-tested with the CAR-derived PR001046 VH-only human IgG1 test antibody and controls (i.e., Rituximab biosimilar, a clinical anti-CD20 IgG1 positive control benchmark that is known to be specific, and a second format-specific, VH-only binder heavy chain antibody (hcAb) control that binds an irrelevant target antigen). This was performed on both fixed and live cells (FIGS. 11A-11D). The results are summarized in Table 13. The PR001046 VH-only human IgG1 test antibody showed a significant specific interaction with BCMA (TNFRSF17), the primary target, on both fixed and live cell microarrays. No other interactions were identified for the CAR-derived PR001046 VH-only human IgG1 test antibody specifically (i.e., absent for Rituximab or the format-specific control), thus indicating high specificity of PR001046 for its primary target, BCMA (TNFRSF17).

TABLE 13
Library and confirmation screen results

Interaction no.

| | | | | | |
|---|---|---|---|---|---|
| 1 | 2 | 3 | 4 | 5 | 6 |

Gene ID

| | | | | | |
|---|---|---|---|---|---|
| | | TNFRSF17 | | | |
| FCGR1A | FCGR2B | (BCMA) | FCGR2A | FCGR3B | FCGR3A |

Unique clone Number (UCN)

| Sample Id | L/C | Rep | 0044 | 0617 | 1720 | 2228 | 2117 | 1968 |
|---|---|---|---|---|---|---|---|---|
| and dose | screen¹ | no. | PM | PM | PM | PM | PM | PM |
| PR001046^{†} | L | 1 | strong | weak | medium | med/strong | weak | weak/med |
| | | 2 | strong | weak | medium | med/strong | v. weak | weak |
| PR001046^{†} | C | 1 | strong | weak | strong** | med/strong | v. weak | weak/med |
| | | 2 | strong | weak | strong** | strong | v. weak | weak/med |
| Negative | C | 1 | med/strong | v. weak | | weak/med | *v. weak | weak |
| control^{††} | | 2 | med/strong | v. weak | | weak/med | v. weak | weak |
| Rituximab | C | 1 | strong | | | weak/med | | *v. weak |
| biosimilar^{‡} | | 2 | medium | | | weak/med | | *v. weak |
| PBS | C | 1 | weak | | | | | |
| (secondary | | 2 | weak | | | | | |
| only) | | | | | | | | |

| | | | Interaction no. | | | | | |
|---|---|---|---|---|---|---|---|---|
| | | | 7 | 8 | 9 | 10 | 11 | 12 |
| | | | Gene ID | | | | | |
| | | | IGHG3 | LDLR | FCGR2A | FCGR2B | SPNS2 | MFSD2B |
| | | | Unique clone Number (UCN) | | | | | |
| Sample Id and | L/C | Rep | 2497 | 2881 | 2685 | 3217 | 6431 | 6466 |
| dose | screen¹ | no. | M | PM | PM | PM | PM | PM |
| PR001046^{†} | L | 1 | strong | v. weak | strong | weak | weak | v. weak |
| | | 2 | strong | v. weak | strong | weak | weak | v. weak |
| PR001046^{†} | C | 1 | strong | *v. weak | med/strong | weak | weak | weak |
| | | 2 | strong | *v. weak | strong | weak | weak | v. weak |
| Negative | C | 1 | strong | | weak/med | v. weak | v. weak | *v. weak |
| control^{††} | | 2 | strong | | medium | v. weak | v. weak | *v. weak |
| Rituximab | C | 1 | med/strong | | medium | *v. weak | v. weak | v. weak |
| biosimilar^{‡} | | 2 | strong | *v. weak | weak/med | *v. weak | v. weak | v. weak |
| PBS (secondary | C | 1 | strong | | | | *v. weak | *v. weak |
| only) | | 2 | strong | | | | v. weak | *v. weak |

| | | | Interaction no. | | | | | |
|---|---|---|---|---|---|---|---|---|
| | | | 13 | 14 | 15 | 16 | 17 | 18 |
| | | | Gene ID | | | | | |
| | | | CXCL12 | IGF2 | IFG1 | IGHG4 | PAPPA | IGHG1 |
| | | | Unique clone Number (UCN) | | | | | |
| Sample Id and | L/C | Rep | 4396 | 4844 | 5073 | 5528 | 5418 | 5548 |
| dose | screen¹ | no. | TS | TS | TS | TS | TS | TS |
| PR001046^{†} | L | 1 | v. weak | v. weak | weak | strong | v. weak | strong |
| | | 2 | v. weak | v. weak | weak | strong | v. weak | strong |
| PR001046^{†} | C | 1 | weak/med | weak | weak/med | strong | v. weak | strong |
| | | 2 | weak | v. weak | weak | strong | *v. weak | strong |
| Negative | C | 1 | v. weak | *v. weak | weak | strong | *v. weak | strong |
| control^{††} | | 2 | weak | *v. weak | weak | strong | | strong |
| Rituximab | C | 1 | weak | weak | weak/med | strong | *v. weak | strong |
| biosimilar^{‡} | | 2 | weak | weak | weak | strong | *v. weak | strong |
| PBS (secondary | C | 1 | v. weak | *v. weak | v. weak | strong | *v. weak | strong |
| only) | | 2 | *v. weak | | | med/strong | *v. weak | strong |

| | | | Interaction no. | | | | |
|---|---|---|---|---|---|---|---|
| | | | 19 | 20 | 21 | 22 | 23 |
| | | | Gene ID | | | | |
| | | | | FCGR3A + | FCGR3A + | | |
| | | | IGHG2 | FCER1G | CD247 | CD20 | EGFR |
| | | | Unique clone Number (UCN) | | | | |
| Sample Id and | L/C | Rep | 5549 | 6071 | 6072 | 0038 | — |
| dose | screen¹ | no. | TS | HD | HD | PM | PM |
| PR001046^{†} | L | 1 | strong | med/strong | weak/med | n/a | n/a |
| | | 2 | strong | med/strong | weak/med | n/a | n/a |
| PR001046^{†} | C | 1 | strong | weak/med | medium | | |
| | | 2 | strong | weak/med | medium | | |
| Negative | C | 1 | strong | v. weak | weak/med | | |
| control^{††} | | 2 | strong | weak | medium | | |
| Rituximab | C | 1 | strong | *v. weak | v. weak | med/strong*** | |
| biosimilar^{‡} | | 2 | strong | *v. weak | v. weak | med/strong*** | |
| PBS (secondary | C | 1 | strong | | | | |
| only) | | 2 | strong | | | | |

^{†}12.5 μg/mL
¹L = library screen; C = confirmation screen
^{††}Negative control = 2.5 μg/mL PR304754
^{‡}1 μg/mL
*interaction can be seen at enhanced contrast
**specific interaction of weak/medium intensity or above
***positive control interaction

All patent publications and non-patent publications are indicative of the level of skill of those skilled in the art to which this disclosure pertains. All these publications (including any specific portions thereof that are referenced) are herein incorporated by reference to the same extent as if each individual publication were specifically and individually indicated as being incorporated by reference.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

What is claimed is:

1. A nucleic acid comprising a sequence that encodes a first chimeric antigen receptor (CAR), wherein the first CAR comprises:
an extracellular domain comprising a first antigen recognition domain consisting of a fully human single variable heavy (VH) domain that binds a first epitope on B-cell maturation antigen (BCMA);
a transmembrane domain; and
an intracellular domain comprising a signaling domain.

2. The nucleic acid of claim 1, wherein the extracellular domain further comprises a second antigen recognition domain consisting of a fully human single variable heavy (VH) domain that binds a second epitope on BCMA, wherein the first and second BCMA epitopes are different.

3. The nucleic acid of claim 2, wherein the first or the second antigen recognition domain has an amino acid sequence of SEQ ID NO: 1, SEQ ID NO: 9, SEQ ID NO: 17 or SEQ ID NO: 25 or a variant of any thereof.

4. (canceled)

5. The nucleic acid of claim 3, wherein the variant of the first or the second antigen recognition domain has an amino acid sequence having at least about 95% identity with any one of SEQ ID NOs: 1, 9, 17, or 25; or
wherein the variant of the first or the second antigen recognition domain has an amino acid sequence having at least about 98% identity with any one of SEQ ID NOs: 1, 9, 17, or 25.

6. (canceled)

7. The nucleic acid of claim 2, wherein the first and second antigen recognition domains are connected by a linker.

8. The nucleic acid of claim 7, wherein the linker comprises an amino acid sequence of GGGX (SEQ ID NO: 33), GGGGX (SEQ ID NO: 34), or GSSGSX (SEQ ID NO: 35), wherein X is either C or S; or
wherein the linker has an amino acid sequence of
(SEQ ID NO: 36)
GGGGSGGGGSGGGGSGGGGS,
(SEQ ID NO: 37)
GSTSGSGKPGSGEGSTKG,
(SEQ ID NO: 38)
KESGSVSSEQLAQFRSLD,
(SEQ ID NO: 39)
EGKSSGSGSESKST,
or
(SEQ ID NO: 40)
GSAGSAAGSGEF.

9. (canceled)

10. The nucleic acid of claim 1, wherein the transmembrane domain is derived from CD3, CD8α, CD28, or CD137; or
wherein the extracellular domain further comprises a hinge domain disposed between the antigen recognition domain and the transmembrane domain; or
wherein the signaling domain comprises a primary signaling domain, co-stimulatory signaling domain, or both a primary signaling domain and a co-stimulatory signaling domain.

11. (canceled)

12. The nucleic acid of claim 10, wherein the hinge domain is derived from IgG1 or IgG4; or
wherein the intracellular domain comprises a CD3ζ primary signaling domain and a 4-1BB co-stimulatory signaling domain, a CD3ζ primary signaling domain and a CD28 co-stimulatory signaling domain, or a CD3ζ primary signaling domain and both 4-1BB and CD28 co-stimulatory signaling domains.

13-17. (canceled)

18. The nucleic acid of claim 1, wherein the first CAR further comprises a signal peptide.

19. The nucleic acid of claim 18, wherein the signal peptide is derived from albumin, CD8a, EPO, IgGκ, or IL-2.

20. The nucleic acid of claim 1, which is codon-optimized for expression in a human immune cell; or
wherein the nucleic acid does not comprise a stretch of 20 or more base pairs of direct or inverted repeat sequences: or
wherein the nucleic acid does not comprise alternate splice sites.

21.-22. (canceled)

23. An expression vector encoding the nucleic acid sequence of claim 1.

24. The expression vector of claim 23, which is a viral vector or a non-viral vector: or wherein the expression vector is encapsulated in a lipid-based carrier.

25. (canceled)

26. The expression vector of claim 24, wherein the non-viral vector is a plasmid.

27. (canceled)

28. A method of producing a genetically modified immune cell or a genetically modified hematopoietic cell, comprising:
introducing the expression vector of claim 23 into an immune cell or a hematopoietic cell.

29. An immune cell or hematopoietic cell comprising the nucleic acid of claim 1.

30. The cell of claim 29, wherein the immune cell is a T cell; or
wherein the immune cell is a NK cell; or
wherein the immune cell is a macrophage or a dendritic cell; or
wherein the immune cell or the hematopoietic cell is derived from a hematopoietic stem cell; or
wherein the immune cell or the hematopoietic cell is derived from an induced pluripotent stem cell; and/or
wherein the immune cell further comprises a second CAR, wherein the second CAR comprises an extracellular domain comprising an antigen recognition domain, a transmembrane domain, and an intracellular signaling domain;
wherein the antigen recognition domain consists of a fully human single VH domain that binds a third BCMA epitope, wherein the first and third BCMA epitopes are different.

31. The cell of claim 30, wherein the T cell is a CD8+ T cell; or wherein the T cell is a central memory T cell or a stem cell-like central memory T cell.

32.-37. (canceled)

38. A pharmaceutical composition comprising a therapeutically effective number of the cells of claim 29.

39. A method of treating cancer or autoimmune disease that involves aberrant BCMA activity, comprising:
administering to a subject in need thereof the pharmaceutical composition of claim 38.

40. The method of claim 39, wherein the cells are allogeneic and have a complete or partial HLA-match with the subject; or
wherein the cells are autologous; and/or
wherein the cancer is a hematological cancer.

41.-42. (canceled)

43. The method of claim 40, wherein the hematological cancer is multiple myeloma.

44. The method of claim 43, further comprising administering to the subject a therapeutically effective amount of an additional active agent that binds a surface antigen on a myeloma cell.

45. The method of claim 44, wherein the additional active agent binds CD3, CD16, CD38, CD44, CD138, CD229, SLAMF7, integrin β7 (ITGB7), Natural Killer Group 2D (NKG2D), NKp44, NKp46, or cereblon E3 ligase; or
wherein the additional active agent is one or more of bortezomib, carfilzomib, ixazomib, lenalidomide, pomalidomide, thalidomide, dexamethasone, prednisone, elotuzumab, daratumumab, isatuximab, and mezigdomide, iberdomide, talquetamab, monalizumab, AMG420, and AMG701.

46. (canceled)

47. The method of claim 40, wherein the cancer is lymphoma.

48. The method of claim 47, further comprising administering to the subject a therapeutically effective amount of an additional active agent that binds a surface antigen on a lymphoma cell.

49. The method of claim 48, wherein the additional active agent is one or more of rituximab, mosunetuzumab, and blinatumomab.

50. The method of claim 39, wherein the autoimmune disease comprises lupus, systemic lupus erythematosus (SLE), myasthenia graves, immune thrombocytopenia (ITP), scleroderma, immune nephritis, Sjogren's syndrome, POEMS syndrome, pemphigus vulgaris, amyloidosis, autoimmune hemolytic anemia, or vasculitis.

51. The method of claim 39, further comprising the step of administering to the subject a therapeutically effective amount of a gamma secretase inhibitor; or
further comprising the step of administering to the subject a therapeutically effective amount of a gamma secretase inhibitor; or
wherein the cancer is relapsed or refractory; or
further comprising administering to the subject lymphodepletion chemotherapy prior to the administering of the cells.

52. The method of claim 50, wherein the gamma secretase inhibitor comprises one or more of Avagacestat, Begacestat, Crenigacestat, Iminostilbene, Itanapraced, Nirogacestat, L-685458, Semagacestat, and Tarenflurbil.

53.-54. (canceled)

55. The method of claim 50, wherein the lymphodepletion chemotherapy comprises administering a therapeutically effective amount of melphalan; or
further comprising performing a stem cell transplant on the subject after the lymphodepletion chemotherapy.

56. (canceled)


SEQUENCE LISTING (91 sequences)

SEQ ID NO: 1 - protein, 122 aa, synthetic construct
EVQLLESGGGLVQPGGSLRLSCAASGFTFSSYAMIWVRQAPGKGLEWVSGISESGGSTYYADSVKGRFTISRDNSKNTLYLQMNSLRAEDTAVYFCVKDLDDILTGYYKDYWGQGTLVTVSS

SEQ ID NO: 2 - protein, 25 aa, synthetic construct
EVQLLESGGGLVQPGGSLRLSCAAS

SEQ ID NO: 3 - protein, 7 aa, synthetic construct
GFTFSSY

SEQ ID NO: 4 - protein, 19 aa, synthetic construct
AMIWVRQAPGKGLEWVSGI

SEQ ID NO: 5 - protein, 6 aa, synthetic construct
SESGGS

SEQ ID NO: 6 - protein, 41 aa, synthetic construct
TYYADSVKGRFTISRDNSKNTLYLQMNSLRAEDTAVYFCVK

SEQ ID NO: 7 - protein, 13 aa, synthetic construct
DLDDILTGYYKDY

SEQ ID NO: 8 - protein, 11 aa, synthetic construct
WGQGTLVTVSS

SEQ ID NO: 9 - protein, 120 aa, synthetic construct
EVQLVETGGGLIQPGGSLRLSCAASGFPVSSNYMSWVRQAPGKGLEWVSVIFSGGRTYYADSVKGRFTISRDNSKNTLYLQMNSLRAEDTAVYYCARRNWIDGRDAFDIWGQGTMVTVSS

SEQ ID NO: 10 - protein, 25 aa, synthetic construct
EVQLVETGGGLIQPGGSLRLSCAAS

SEQ ID NO: 11 - protein, 7 aa, synthetic construct
GFPVSSN

SEQ ID NO: 12 - protein, 19 aa, synthetic construct
YMSWVRQAPGKGLEWVSVI

SEQ ID NO: 13 - protein, 5 aa, synthetic construct
FSGGR

SEQ ID NO: 14 - protein, 41 aa, synthetic construct
TYYADSVKGRFTISRDNSKNTLYLQMNSLRAEDTAVYYCAR

SEQ ID NO: 15 - protein, 12 aa, synthetic construct
RNWIDGRDAFDI

SEQ ID NO: 16 - protein, 11 aa, synthetic construct
WGQGTMVTVSS

SEQ ID NO: 17 - protein, 120 aa, synthetic construct
EVQLVETGGGLIQPGGSLRLSCAASGFTVSSNYMTWVRQAPGKGLEWVSVIFSGGSTYYADSVKGRFTISRDNSKNTLYLQMNSLRAEDTAVYYCARRIAVAGHDVFDIWGQGTMVTVSS

SEQ ID NO: 18 - protein, 25 aa, synthetic construct
EVQLVETGGGLIQPGGSLRLSCAAS

SEQ ID NO: 19 - protein, 7 aa, synthetic construct
GFTVSSN

SEQ ID NO: 20 - protein, 19 aa, synthetic construct
YMTWVRQAPGKGLEWVSVI

SEQ ID NO: 21 - protein, 5 aa, synthetic construct
FSGGS

SEQ ID NO: 22 - protein, 41 aa, synthetic construct; identical to SEQ ID NO: 14

SEQ ID NO: 23 - protein, 12 aa, synthetic construct
RIAVAGHDVFDI

SEQ ID NO: 24 - protein, 11 aa, synthetic construct
WGQGTMVTVSS

SEQ ID NO: 25 - protein, 122 aa, synthetic construct
EVQLVETGGGLIQPGGSLRLSCAASGFTVSDNYMTWVRQAPGKGLEWVSVIFSGGNTYYADSVKGRFTISRDNAKNTLYLQMNSLRAEDTALYYCARRNYDDTRGTDVFDIWGQGTMVTVSS

SEQ ID NO: 26 - protein, 25 aa, synthetic construct
EVQLVETGGGLIQPGGSLRLSCAAS

SEQ ID NO: 27 - protein, 7 aa, synthetic construct
GFTVSDN

SEQ ID NO: 28 - protein, 19 aa, synthetic construct
YMTWVRQAPGKGLEWVSVI

SEQ ID NO: 29 - protein, 5 aa, synthetic construct
FSGGN

SEQ ID NO: 30 - protein, 41 aa, synthetic construct
TYYADSVKGRFTISRDNAKNTLYLQMNSLRAEDTALYYCAR

SEQ ID NO: 31 - protein, 14 aa, synthetic construct
RNYDDTRGTDVFDI

SEQ ID NO: 32 - protein, 11 aa, synthetic construct
WGQGTMVTVSS

SEQ ID NO: 33 - protein, 5 aa, synthetic construct
GGGGX

SEQ ID NO: 34 - protein, 6 aa, synthetic construct
GSSGSX

SEQ ID NO: 35 - protein, 20 aa, synthetic construct
GGGGSGGGGSGGGGSGGGGS

SEQ ID NO: 36 - protein, 18 aa, synthetic construct
GSTSGSGKPGSGEGSTKG

SEQ ID NO: 37 - protein, 18 aa, synthetic construct
KESGSVSSEQLAQFRSLD

SEQ ID NO: 38 - protein, 14 aa, synthetic construct
EGKSSGSGSESKST

SEQ ID NO: 39 - protein, 12 aa, synthetic construct
GSAGSAAGSGEF

SEQ ID NO: 40 - protein, 21 aa, Homo sapiens
LCYLLDGILFIYGVILTALFL

SEQ ID NO: 41 - protein, 22 aa, Homo sapiens
MALIVLGGVAGLLLFIGLGIFF

SEQ ID NO: 42 - protein, 24 aa, Homo sapiens
IYIWAPLAGTCGVLLLSLVITLYC

SEQ ID NO: 43 - protein, 27 aa, Homo sapiens
FWVLVVVGGVLACYSLLVTVAFIIFWV

SEQ ID NO: 44 - protein, 27 aa, Homo sapiens
IISFFLALTSTALLFLLFFLTLRFSVV

SEQ ID NO: 45 - protein, 9 aa, Homo sapiens
QSFGLLDPK

SEQ ID NO: 46 - protein, 23 aa, Homo sapiens
LSEGDKVKMDSRIQVLSRGVNQT

SEQ ID NO: 47 - protein, 49 aa, Homo sapiens
KPTTTPAPRPPTPAPTIASQPLSKRPEACRPAAGGAVHTRGLDFACDIY

SEQ ID NO: 48 - protein, 39 aa, Homo sapiens
IEVMYPPPYLDNERSNGTIIHVKGKHLCPSPLFPGPSKP

SEQ ID NO: 49 - protein, 22 aa, Homo sapiens
EPKSCDKTHTCPPCPAPELLGG

SEQ ID NO: 50 - protein, 18 aa, Homo sapiens
ERKCCVECPPCPAPPAAA

SEQ ID NO: 51 - protein, 19 aa, Homo sapiens
ESKYGPPCPPCPAPEFLGG

SEQ ID NO: 52 - protein, 112 aa, Homo sapiens
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - protein, 55 aa, Homo sapiens
KNRKAKAKPVTRGAGAGGRQRGQNKERPPPVPNPDYEPIRKGQRDLYSGLNQRRI

SEQ ID NO: 55 - protein, 41 aa, Homo sapiens
RSKRSRLLHSDYMNMTPRRPGPTRKHYQPYAPPRDFAAYRS

SEQ ID NO: 56 - protein, 48 aa, Homo sapiens
QRRKYRSNKGESPVEPAEPCHYSCPREEEGSTIPIQEDYRKPEPACSP

SEQ ID NO: 57 - protein, 25 aa, Homo sapiens
TYCFAPRCRERRRNERLRRESVRPV

SEQ ID NO: 58 - protein, 22 aa, Homo sapiens
MIETYNQTSPRSAATGLPISMK

SEQ ID NO: 59 - protein, 25 aa, Homo sapiens
TYCFAPRCRERRRNERLRRESVRPV

SEQ ID NO: 60 - protein, 61 aa, Homo sapiens
KWKKKKRPRNSYKCGTNTMEREESEQTKKREKIHIPERSDEAQRVFKSSKTSSCDKSDTCF

SEQ ID NO: 61 - protein, 66 aa, Homo sapiens
KRGRKKLLYIFKQPFMRPVQTTQEEDGCSCRFPEEEEGGCELLCARPRRSPAQEDGKVYINMPGRG

SEQ ID NO: 62 - protein, 38 aa, Homo sapiens
CWLTKKKYSSSVHDPNGEYMFMRAVNTAKKSRLTDVTL

SEQ ID NO: 63 - protein, 52 aa, Homo sapiens
YFLGRLVPRGRGAAEAATRKQRITETESPYQELQGQRSDVYSDLNTQRPYYK

SEQ ID NO: 64 - protein, 42 aa, Homo sapiens
RLKIQVRKAAITSYEKSDGVYTGLSTRNQETYETLKHEKPPQ

SEQ ID NO: 65 - protein, 41 aa, Homo sapiens; identical to SEQ ID NO: 55

SEQ ID NO: 66 - protein, 39 aa, Homo sapiens
HRWCSNKKNAAVMDQEPAGNRTVNSEDSDEQDHQEVSYA

SEQ ID NO: 67 - protein, 296 aa, Homo sapiens
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 68 - protein, 42 aa, Homo sapiens
ALYLLRRDQRLPPDAHKPPGGGSFRTPIQEEQADAHSTLAKI

SEQ ID NO: 69 - protein, 619 aa, Homo sapiens
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 70 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 71 - protein, 123 aa, Homo sapiens
MLRLLLALNLFPSIQVTGNKILVKQSPMLVAYDNAVNLSWKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 72 - protein, 101 aa, Homo sapiens
MLRLLLALNLFPSIQVTGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 73 - protein, 234 aa, Homo sapiens
MPCGLSALIMCPKGMVAVVVAVDDGDSQALAGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 74 - protein, 18 aa, Homo sapiens
MKWVTFISLLFLFSSAYS

SEQ ID NO: 75 - protein, 19 aa, synthetic construct
MKWVTFISLLFLFSSSSRA

SEQ ID NO: 76 - protein, 21 aa, Homo sapiens
MALPVTALLLPLALLLHAARP

SEQ ID NO: 77 - protein, 16 aa, Homo sapiens
MPLLLLLPLLWAGALA

SEQ ID NO: 78 - protein, 27 aa, Homo sapiens
MGVHECPAWLWLLLSLLSLPLGLPVLG

SEQ ID NO: 79 - protein, 20 aa, Homo sapiens
MYRMQLLSCIALSLALVTNS

SEQ ID NO: 80 - protein, 20 aa, Mus musculus
METDTLLLWVLLLWVPGSTG

SEQ ID NO: 81 - protein, 20 aa, Homo sapiens
MEAPAQLLFLLLLWLPDTTG

SEQ ID NO: 82 - protein, 20 aa, Homo sapiens
MVLQTQVFISLLLWISGAYG

SEQ ID NO: 83 - protein, 20 aa, synthetic construct
MEAPAQLLFLLLLWLPSSRA

SEQ ID NO: 84 - protein, 23 aa, Homo sapiens
MDAMKRGLCCVLLLCGAVFVSPS

SEQ ID NO: 85 - protein, 17 aa, Homo sapiens
MLLLLLLLGLRLQLSLG

SEQ ID NO: 86 - protein, 16 aa, synthetic construct
MLLLLLLLLLLALALA

SEQ ID NO: 87 - protein, 21 aa, synthetic construct
MWWRLWWLLLLLLLLWPMVWA

SEQ ID NO: 88 - DNA, 366 bp, synthetic construct
  source 1..366: mol_type = other DNA
gaggtgcagctgctggagagcggcggcggcctggtgcagcccggcggcagcctgaggctgagctgcgccgccagcggcttcaccttcagcagctacgccatgatctgggtgaggcaggcccccggcaagggcctggagtgggtgagcggcatcagcgagagcggcggcagcacctactacgccgacagcgtgaagggcaggttcaccatcagcagggacaacagcaagaacaccctgtacctgcagatgaacagcctgagggccgaggacaccgccgtgtacttctgcgtgaaggacctggacgacatcctgaccggctactacaaggactactggggccagggcaccctggtgaccgtgagcagc

SEQ ID NO: 89 - DNA, 360 bp, synthetic construct
  source 1..360: mol_type = other DNA
gaggtgcagctggtggagaccggcggcggcctgatccagcccggcggcagcctgaggctgagctgcgccgccagcggcttccccgtgagcagcaactacatgagctgggtgaggcaggcccccggcaagggcctggagtgggtgagcgtgatcttcagcggcggcaggacctactacgccgacagcgtgaagggcaggttcaccatcagcagggacaacagcaagaacaccctgtacctgcagatgaacagcctgagggccgaggacaccgccgtgtactactgcgccaggaggaactggatcgacggcagggacgccttcgacatctggggccagggcaccatggtgaccgtgagcagc

SEQ ID NO: 90 - DNA, 360 bp, synthetic construct
  source 1..360: mol_type = other DNA
gaggtgcagctggtggagaccggcggcggcctgatccagcccggcggcagcctgaggctgagctgcgccgccagcggcttcaccgtgagcagcaactacatgacctgggtgaggcaggcccccggcaagggcctggagtgggtgagcgtgatcttcagcggcggcagcacctactacgccgacagcgtgaagggcaggttcaccatcagcagggacaacagcaagaacaccctgtacctgcagatgaacagcctgagggccgaggacaccgccgtgtactactgcgccaggaggatcgccgtggccggccacgacgtgttcgacatctggggccagggcaccatggtgaccgtgagcagc

SEQ ID NO: 91 - DNA, 360 bp, synthetic construct; identical to SEQ ID NO: 90
  source 1..360: mol_type = other DNA